(19) United States
(12) Patent Application Publication
(10) Pub. No.: US 2020/0206303 A1
(43) Pub. Date: Jul. 2, 2020

(54) METHODS AND COMPOSITIONS FOR DETECTING AND TREATING ENDOMETRIOSIS

(71) Applicants: Dot Laboratories, Inc., San Francisco, CA (US); Yale University, New Haven, CT (US)

(72) Inventors: Heather BOWERMAN, San Francisco, CA (US); Hugh TAYLOR, Easton, CT (US)

(21) Appl. No.: 16/798,077

(22) Filed: Feb. 21, 2020

Related U.S. Application Data
(63) Continuation of application No. PCT/US18/48649, filed on Aug. 29, 2018.
(60) Provisional application No. 62/552,365, filed on Aug. 30, 2017.

(51) Int. Cl. A61K 38/09; C12Q 1/6883; G16B 20/00; A61P 15/00; A61K 31/513

(52) U.S. Cl. CPC A61K 38/09; C12Q 1/6883; G16B 20/00; C12Q 2600/158; A61K 31/513; C12Q 2600/178; A61P 15/00

(57) ABSTRACT

The present disclosure provides improved methods of providing endometriosis testing to patients, as well as improved methods of monitoring and adjusting endometriosis treatments.

### CROSS-REFERENCE STATEMENT

This application is a continuation of PCT Application No. PCT/US18/48649, filed Aug. 29, 2018; which claims the benefit of U.S. Provisional Application Ser. No. 62/552,365 filed on Aug. 30, 2017; which are incorporated by reference herein in their entirety.

### BACKGROUND OF THE INVENTION

Endometriosis is a common condition affecting women of pubescent and reproductive age. The disease is thought to be caused by endometrial tissue which migrates from its normal position lining the uterus to other parts of the body, primarily within the abdominal cavity. The ovaries and gut wall are commonly affected. The displaced endometrial tissue, like that in its normal position, grows and declines according to the menstrual cycle as a result of the actions of the ovarian hormones. Endometriosis may cause many symptoms including, but not limited to, abdominal pain, gastrointestinal upset, excessive bleeding, infertility and menstrual disturbance.

First-line treatments for endometriosis may either manage pain without affecting the disease process per se (e.g. NSAIDS), or may ultimately prove ineffective in certain patients (e.g., progestins, which are ineffective in suppressing endometriosis in a subgroup of women whose endometrial tissue does not respond normally to progesterone). Second-line therapies, such as Gonadotropin-releasing Hormone (GnRH) agonists or antagonists, are associated with varying degrees of unpleasant side effects, and thus require accurate management of dosing to manage side effects.

### SUMMARY OF THE INVENTION

This disclosure addresses, among other things, a need in the art for minimally-invasive, accurate and more efficient methods of detecting, diagnosing, monitoring, and treating endometriosis. In one aspect, the present disclosure provides a method of identifying and treating endometriosis in a female subject, comprising: (a) obtaining a fluid sample from the female subject, wherein the fluid sample comprises ribonucleic acids (RNA); (b) determining an expression level of at least one miRNA or at least one non-coding RNA (ncRNA) from the fluid sample from the subject, wherein the at least one miRNA or the at least one ncRNA is associated with endometriosis; (c) diagnosing endometriosis in the subject based on the expression level of the at least one miRNA or the at least one ncRNA; and (d) administering an initial dose regimen of a Gonadotropin-releasing hormone (GnRH) antagonist to the subject in order to treat the endometriosis diagnosed in the subject in (c). In some embodiments, the fluid sample comprises at least one miRNA. In some embodiments, the fluid sample is blood, saliva, menstrual blood, or menstrual effluent. In some embodiments, the female subject is receiving treatment for endometriosis, and the endometriosis diagnosed and treated is refractory endometriosis. In some embodiments, the treatment is progestin therapy. In some embodiments, the progestin therapy is dydrogesterone, medroxyprogesterone acetate, depot medroxyprogesterone acetate, norethisterone, or an oral contraceptive pill. In some embodiments, the subject is experiencing symptoms associated with endometriosis. In some embodiments, the subject is experiencing one or more of dysmennorhea, pain with bowel movements or urination, or excessive bleeding. In some embodiments, the subject is not experiencing symptoms associated with endometriosis. In some embodiments, the at least one miRNA is selected from the group consisting of let-7a, let-7b, let-7c, let-7d, let-7e, let-7f, miR-135a, miR-135b, miR-18a, miR-125b, miR-143, miR-145, miR-150, miR-342, miR-451a, miR-500a, miR-3613, and miR-6755, or any combination thereof. In some embodiments, the at least one miRNA is selected from the group consisting of let-7c, let-7d, let-7f, miR-18a, miR-125b, miR-143, miR-150, miR-342, miR-451a, miR-500a, miR-3613, and miR-6755, or any combination thereof. In some embodiments, the at least one miRNA is selected from the group consisting of let-7a, let-7b, let-7c, let-7d, let-7e, let-7f, mir135a, and mir135b, or any combination thereof. In some embodiments, the at least one miRNA is selected from the group consisting of miR-125b, miR-150, miR-342, miR-451a, miR-3613, and let-7b, or any combination thereof. In some embodiments, the at least one miRNA is selected from the group consisting of miR-150, 451a, and 3613, or any combination thereof. In some embodiments, the method further comprises repeating (a)-(c) and adjusting the initial dose regimen of the GnRH antagonist when endometriosis is not detected. In some embodiments, the method further comprises repeating (a)-(c) and adjusting the initial dose regimen of the GnRH antagonist when endometriosis is detected. In some embodiments, the method further comprises repeating (a)-(c) and terminating administration of the GnRH antagonist when endometriosis is not detected. In some embodiments, the method comprises repeating (a)-(c) every 1 month, 6 months, or 1 year. In some embodiments, the GnRH antagonist is goserelin acetate, buserelin, histrelin, deslorelin, nafarelin, and triptorelin, leuproreolin, or Elagolix. In some embodiments, the sample is menstrual blood or menstrual effluent and the menstrual blood or menstrual effluent is collected by the subject using a menstrual cup. In some embodiments, the sample is saliva and the saliva was collected by the subject using a home saliva sampling kit.

In one aspect, the present disclosure also provides a method of identifying and treating endometriosis in a female subject, comprising; (a) receiving information characterizing an expression level of at least one miRNA or non-coding RNA (ncRNA) from a fluid sample from the female subject; (b) diagnosing endometriosis in the subject based on the expression level of the at least one miRNA or the at least one ncRNA from the fluid sample from the female subject; and (c) administering an initial dose regimen of a Gonadotropin-releasing hormone (GnRH) antagonist to the female subject in order to treat the endometriosis diagnosed in the female subject in (b). In some embodiments, the at least one miRNA is selected from the group consisting of let-7a, let-7b, let-7c, let-7d, let-7e, let-7f, miR-135a, miR-135b, miR-18a, miR-125b, miR-143, miR-145, miR-150, miR-342, miR-451a, miR-500a, miR-3613, and miR-6755, or any combination thereof. In some embodiments, the method further comprises repeating (a)-(b) and adjusting the initial dose regimen of the GnRH antagonist when endometriosis is not detected. In some embodiments, the method further comprises repeating (a)-(b) and adjusting the initial dose regimen of the GnRH antagonist when endometriosis is detected. In some embodiments, the method further comprises repeating (a)-(b) and terminating administration of the GnRH antagonist when endometriosis is not detected. In some embodiments, the method comprises repeating (a)-(b) every 1 month, 6 months, or 1 year. In some embodiments, the GnRH antagonist is goserelin acetate, buserelin, histrelin, deslorelin, nafarelin, triptorelin, leuproreolin, or Elagolix. In some embodiments, the fluid sample is blood, plasma, serum, saliva, menstrual blood, or menstrual effluent.

In another aspect, the present disclosure also provides a method of treating endometriosis in a female subject, comprising administering to the female subject an initial dose regimen of a Gonadotropin-releasing hormone (GnRH) antagonist, wherein a fluid sample from the female subject has a detected level of at least one miRNA or at least one ncRNA associated with endometriosis. In some embodiments, the fluid sample is blood, plasma, serum, saliva, menstrual blood or menstrual effluent. In some embodiments, the at least one miRNA is selected from the group consisting of let-7a, let-7b, let-7c, let-7d, let-7e, let-7f, miR-135a, miR-135b, miR-18a, miR-125b, miR-143, miR-145, miR-150, miR-342, miR-451a, miR-500a, miR-3613, and miR-6755, or any combination thereof. In some embodiments, the GnRH antagonist is goserelin acetate, buserelin, histrelin, deslorelin, nafarelin, and triptorelin, leuproreolin, or Elagolix. In some embodiments, the endometriosis is refractory endometriosis. In some embodiments, the female subject is receiving a progestin therapy and the endometriosis is refractory endometriosis.

In one aspect, the present disclosure also provides a method of treating endometriosis in a subject in need thereof comprising: administering to the subject in need thereof an initial dose of a Gonadotropin-releasing hormone (GnRH)-antagonist; monitoring the level of at least one miRNA or at least one non-coding RNA (ncRNA) associated with endometriosis in the subject in need thereof over time; and adjusting the initial dose of said GnRH-antagonist when the level of the at least one miRNA or the at least one ncRNA associated with endometriosis increases or decreases over time. In some embodiments, the GnRH antagonist is goserelin acetate, buserelin, histrelin, deslorelin, nafarelin, and triptorelin, leuproreolin, or Elagolix. In some embodiments, the at least one miRNA is selected from the group consisting of let-7a, let-7b, let-7c, let-7d, let-7e, let-7f, miR-135a, miR-135b, miR-18a, miR-125b, miR-143, miR-145, miR-150, miR-342, miR-451a, miR-500a, miR-3613, and miR-6755, or any combination thereof. In some embodiments, the monitoring comprises (a) determining an expression level of at least one miRNA or the at least one ncRNA from a fluid sample from the subject; or (b) receiving information characterizing the expression level of at least one miRNA or the at least one ncRNA from a fluid sample from the female subject. In some embodiments, the method comprises adjusting the initial dose of GnRH antagonist when the levels of at least one of miR-3613 or let-7b decrease over time. In some embodiments, the method comprises adjusting the initial dose of GnRH when the levels of at least one of miR-125b, miR-150, miR-342, or miR-451a increase over time. In some embodiments, the time over which the level of the at least one miRNA or the at least one ncRNA increases or decreases is 1 month, 6 months, or 1 year.

In one aspect, the present disclosure also provides a method of detecting miRNA or non-coding RNA (ncRNA) in a female subject suspected of having endometriosis, comprising detecting at least one miRNA or at least one ncRNA from a fluid sample from the female subject suspected of having endometriosis, wherein the fluid sample comprises menstrual effluent or menstrual blood. In some embodiments, the method further comprises administering an initial dose regimen of a treatment for endometriosis to the female subject suspected of having endometriosis. In some embodiments, the treatment for endometriosis comprises a GnRH antagonist. In some embodiments, the at least one miRNA is selected from the group consisting of let-7a, let-7b, let-7c, let-7d, let-7e, let-7f, miR-135a, miR-135b, miR-18a, miR-125b, miR-143, miR-145, miR-150, miR-342, miR-451a, miR-500a, miR-3613, and miR-6755, or any combination thereof. In some embodiments, the method comprises repeating the detecting of the at least one miRNA or the at least one ncRNA every 1 month, 6 months, or 1 year. In some embodiments, the method further comprises diagnosing endometriosis in the subject suspected of having endometriosis based on an expression level of the at least one miRNA or the at least one ncRNA from the fluid sample from the female subject and administering to the female subject a treatment for endometriosis.

In one aspect, the present disclosure also provides a method of treating endometriosis in a female subject, comprising administering to the female subject an initial dose regimen of an endometriosis treatment when a menstrual blood or menstrual effluent sample from the female subject has a level of at least one miRNA or at least one ncRNA associated with endometriosis. In some embodiments, the at least one miRNA is selected from the group consisting of let-7a, let-7b, let-7c, let-7d, let-7e, let-7f, miR-135a, miR-135b, miR-18a, miR-125b, miR-143, miR-145, miR-150, miR-342, miR-451a, miR-500a, miR-3613, and miR-6755, or any combination thereof. In some embodiments, the endometriosis is refractory endometriosis. In some embodiments, the female subject is receiving a progestin therapy and the endometriosis is refractory endometriosis.

In one aspect, the present disclosure also provides a method of conducting a diagnostic test on a subject and providing results to a medical care provider of the subject, comprising: (a) providing to the subject a saliva, menstrual blood, or menstrual effluent sampling kit, wherein the saliva, menstrual blood, or menstrual effluent sampling kit comprises: (i) a saliva, menstrual blood, or menstrual effluent recovery and collection device; and (ii) a code which uniquely identifies the saliva, menstrual blood, or menstrual effluent recovery and collection device; (b) assigning, in a first database, a code which uniquely identifies the subject; (c) receiving separately from the subject (i) a saliva, menstrual blood, or menstrual effluent sample in the saliva, menstrual blood, or menstrual effluent; (ii) the code which uniquely identifies the saliva, menstrual blood, or menstrual effluent sampling kit; and (iii) a pre-assigned code which uniquely identifies the medical care provider of the subject; (d) associating, in a second database, the code which uniquely identifies the subject with the code which uniquely identifies the saliva, menstrual blood, or menstrual effluent and the pre-assigned code which uniquely identifies the medical care provider of the subject; (e) processing the saliva sample in the saliva, menstrual blood, or menstrual effluent to determine an expression level of at least one miRNA or at least one ncRNA; and (0 entering the expression level of at least one miRNA or the at least one ncRNA from the processed saliva, menstrual blood, or menstrual effluent sample into a third database and associating the expression level of at least one miRNA with the subject and the medical care provider of the subject via the association created in (d), wherein the expression level of at least one miRNA or of the at least one ncRNA in the database is accessible via a web-based interface by the subject and the medical care provider of the subject. In some embodiments, the first, second, and third databases are a single database. In some embodiments, the first, second, and third databases are separate databases. In some embodiments, (a) comprises mailing the saliva sampling kit to the subject at a home address or preferred mailing address of the subject. In some embodiments, (a) comprises mailing the saliva, menstrual blood, or menstrual effluent sampling kit to the medical care provider of the subject. In some embodiments, (a) comprises billing the saliva, menstrual blood, or menstrual effluent testing kit to a credit card of the subject. In some embodiments, the code that uniquely identifies the saliva, menstrual blood, or menstrual effluent sampling kit is provided by the medical care provider of the subject via a web interface. In some embodiments, the code that uniquely identifies the saliva, menstrual blood, or menstrual effluent sampling kit is provided by the subject via a web interface. In some embodiments, receiving the saliva, menstrual blood, or menstrual effluent sampling kit from the subject in (c) comprises receiving the saliva, menstrual blood, or menstrual effluent sampling kit via mail from the subject's home address or preferred mailing address. In some embodiments, receiving the saliva sample in the saliva, menstrual blood, or menstrual effluent sampling kit from the subject in (c) comprises receiving the saliva, menstrual blood, or menstrual effluent sampling kit via mail from a workplace address of the medical care provider of the subject. In some embodiments, the method further comprises providing a clinical indication based on the expression level of the at least one miRNA or the at least one ncRNA, wherein the clinical indication is also accessible via the web-based interface by the subject and the medical care provider of the subject. In some embodiments, the clinical indication is endometriosis. In some embodiments, the processing the saliva, menstrual blood, or menstrual effluent sample in the saliva, menstrual blood, or menstrual effluent sampling kit to determine an expression level of the at least one miRNA or the at least one ncRNA in (e) comprises sending the saliva sampling kit to a third party diagnostic laboratory to determine an expression level of the at least one miRNA or the at least one ncRNA.

In one aspect, the present disclosure also provides a method of conducting a diagnostic test for endometriosis on a subject and providing results to the subject and the medical care provider of the subject, comprising: (a) assigning, in a first database, a code which uniquely identifies the subject; (b) receiving from the subject (i) a stabilized fluid sample; (ii) the code which uniquely identifies the stabilized fluid sample; and (iii) a pre-assigned code which uniquely identifies the medical care provider of the subject; (c) associating, in a second database, the code which uniquely identifies the subject with the code which uniquely identifies the fluid sample and the pre-assigned code which uniquely identifies the medical care provider of the subject; (d) processing the fluid sample to determine an expression level of at least one miRNA or at least one non-coding RNA (ncRNA); and (e) entering the expression level of the at least one miRNA or the at least one ncRNA from the processed fluid sample into a third database and associating the expression level of at least one miRNA or the at least one ncRNA with the subject and the medical care provider of the subject via the association created in (d), wherein the expression level of at least one miRNA or the at least one ncRNA in the database is accessible via a web-based interface by the subject and the medical care provider of the subject. In some embodiments, the fluid sample is a saliva, menstrual blood, or menstrual effluent sample. In some embodiments, the fluid sample is a menstrual blood or menstrual effluent sample. In some embodiments, the menstrual blood or menstrual effluent sample is stabilized by spotting and drying on paper. In some embodiments, the menstrual blood or menstrual effluent sample is stabilized by addition of RNase inhibitor.

In one aspect, the disclosure provides a method of conducting a diagnostic test on a subject and providing results to the subject and the subject's physician, comprising: (a) providing to the subject a saliva sampling kit, wherein the saliva sampling kit comprises: (i) a saliva recovery and collection device; and (ii) a code which uniquely identifies the saliva recovery and collection device; (b) assigning, in a first database, a code which uniquely identifies the subject; (c) receiving separately from the subject (i) a saliva sample in the saliva sampling kit; (ii) the code which uniquely identifies the saliva sampling kit; and (iii) a pre-assigned code which uniquely identifies the patient's physician; (d) associating, in a second database, the code which uniquely identifies the subject with the code which uniquely identifies the saliva sampling kit and the pre-assigned code which uniquely identifies the subject's physician; (e) processing the saliva sample in the saliva sampling kit to determine an expression level of at least one miRNA; and (0 entering the expression level of at least one miRNA from the processed saliva sample into a third database and associating the expression level of at least one miRNA with the subject and the subject's physician via the association created in (d), wherein the expression level of at least one miRNA in the database is accessible via a web-based interface by the subject and the subject's physician. In some embodiments, the first, second, and third databases are a single database. In some embodiments, the first, second, and third databases are separate databases. In some embodiments, (a) comprises mailing to the subject at their home address the saliva sampling kit. In some embodiments, (a) comprises mailing to the subject's physician the saliva sampling kit. In some embodiments, (a) comprises billing the cost of the saliva testing kit to the patient's credit card. In some embodiments, the code that uniquely identifies the saliva sampling kit is provided by the subject's physician via a web interface. In some embodiments, the code that uniquely identifies the saliva sampling kit is provided by the subject via a web interface. In some embodiments, receiving the saliva sample in the saliva sampling kit from the subject in (c) comprises receiving the saliva sampling kit via mail from the patient's home address. In some embodiments, receiving the saliva sample in the saliva sampling kit from the subject in (c) comprises receiving the saliva sampling kit via mail from the patient's physician's workplace address. In some embodiments, the method further comprises providing a clinical indication based on the expression level of at least one miRNA, wherein the diagnostic indication is also accessible via the web-based interface by the subject and the subject's physician. In some embodiments, the clinical indication is endometriosis. In some embodiments, the processing the saliva sample in the saliva sampling kit to determine an expression level of at least one miRNA in (e) comprises sending the saliva sampling kit to a third party diagnostic laboratory to determine an expression level of at least one miRNA.

In another aspect, the disclosure provides a method of identifying and treating refractory endometriosis in a female subject receiving progestin therapy, comprising: (a) obtaining a fluid sample from the subject, wherein the fluid sample comprises ribonucleic acids such as miRNA and the subject is receiving progestin therapy for endometriosis; (b) determining an expression level of at least one miRNA corresponding to the ribonucleic acids from the saliva sample from the subject, wherein the at least one miRNA is associated with endometriosis; (c) diagnosing endometriosis in the subject based on the expression level of the at least one miRNA; and (d) administering an initial dose regimen of a Gonadotropin-releasing Hormone (GnRH) antagonist to the subject in order to treat the endometriosis diagnosed in the subject in (c).

In another aspect, the invention provides a method of identifying and treating refractory endometriosis in a female subject in need thereof, comprising: (a) obtaining a fluid sample from the subject, wherein the fluid sample comprises ribonucleic acids; (b) determining an expression level of at least one miRNA corresponding to the ribonucleic acids from the saliva sample from the subject, wherein the at least one miRNA is associated with treatment-resistant endometriosis; (c) diagnosing treatment-resistant endometriosis in the subject based on the expression level of the at least one miRNA; and (d) administering an initial dose regimen of a GnRH antagonist to the subject in order to treat the treatment-resistant endometriosis diagnosed in the subject in (c). In one embodiment, the subject is experiencing symptoms associated with endometriosis. In some embodiments, the subject is experiencing one or more of dysmennorhea, pain with bowel movements or urination, or excessive bleeding. In some embodiments, the subject is not experiencing symptoms associated with endometriosis. In some embodiments, the fluid sample is blood, serum, saliva, menstrual blood or menstrual effluent. In another embodiment, the at least one miRNA is selected from the group consisting of let-7, miR-125, miR-150, miR-342, miR-145, miR-143, miR-500, miR-451, miR-18, miR-6755, and miR-3613, and any combination thereof. In some embodiments, the method further comprises repeating (a)-(c) and adjusting the initial dose regimen of the GnRH antagonist when endometriosis is not detected. In some embodiments, the method further comprises repeating (a)-(c) and terminating administration of the GnRH antagonist when endometriosis is not detected. In some embodiments, the method further comprises repeating (a)-(c) every day, every week, every month, every 2 months, every 3 months, every 6 months, every year, every other year, or other period of time. In some embodiments, the progestin therapy is dydrogesterone, medroxyprogesterone acetate, depot medroxyprogesterone acetate, norethisterone, or an oral contraceptive pill. In some embodiments, the GnRH antagonist is goserelin acetate, buserelin, histrelin, deslorelin, nafarelin, and triptorelin, leuproreolin, or Elagolix.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A and 1B depict methods of using biomarkers and kits as provided herein. FIG. 1A depicts different stages in the presentation or diagnosis of endometriosis at which kits or biomarkers as provided herein can be used. Biomarkers as used herein may predict future onset of endometriosis. Kits in combination with non-coding RNA (e.g., miRNA) testing as described herein can be used to identify endometriosis and obviate diagnostic laparoscopic surgery in patients. Kits in combination with non-coding RNA (e.g., miRNA) testing as described herein can also be used to inform future treatment in diagnosed individuals (e.g. administration of second-line agents such as Gonadotropin-releasing Hormone (GnRH) modulators). FIG. 1B depicts a workflow by which a saliva collection kit can be provided to a patient, sample processed, and results of a non-coding RNA (e.g., miRNA) test made available.

FIGS. 2 and 3 depict exemplary reports that can be provided to a patient, caregiver (e.g. family member, home health aide), or medical care provider (e.g. physician) as part of the methods described herein.

FIG. 4 depicts methods by which a saliva/menstrual effluent sampling kit to evaluate ncRNA (e.g., miRNA) expression or any of the clinical indications described herein can be provided to a patient, and methods by which the kit can be received from the patient or the patient's medical care provider (e.g. physician).

FIG. 5 depicts an exemplary web portal for a patient.

FIG. 6 depicts an exemplary web portal for a patient's medical care provider (e.g. physician).

FIG. 7 is an image depicting the result of relative expression of miRNAs in saliva in endometriosis and control groups.

FIG. 8 is an image depicting the result of relative expression of miRNAs in saliva in endometriosis and control groups via an alternate representation scheme.

FIG. 9 is a diagram depicting an exemplary computer system for executing methods according to the disclosure.

FIGS. 10A, 10B, and 10C depict exemplary formats for documentation for a test to be provided to the subject. FIG. 10A is an image depicting a paper order form for the medical care provider (e.g. physician) to order the test. The form may be an alternative to a digital or web-based order flow via the physician portal. FIGS. 10B and 10C show two parts of an informed consent form that a patient may be required to review and sign prior to providing a saliva, blood serum, blood plasma, menstrual blood, menstrual effluent, urine, or other body fluid sample.

### DETAILED DESCRIPTION OF THE INVENTION

Overview

The present disclosure provides novel methods for characterizing, monitoring, and analyzing samples from patients having endometriosis, at risk of having endometriosis, or suspected of having endometriosis. Generally, the methods provided herein involve the detection or quantitation of biomarkers in a sample from a subject, particularly non-coding RNA (e.g., miRNA). Also provided are methods of treating patients (e.g., with a Gonadotropin-releasing Hormone (GnRH) antagonist or agonist) monitored or analyzed by these methods, and related kits, compositions and systems, for performing these methods—particularly in a minimally-invasive and routine manner. Also provided are methods of detecting, diagnosing, monitoring, or predicting endometriosis in a subject.

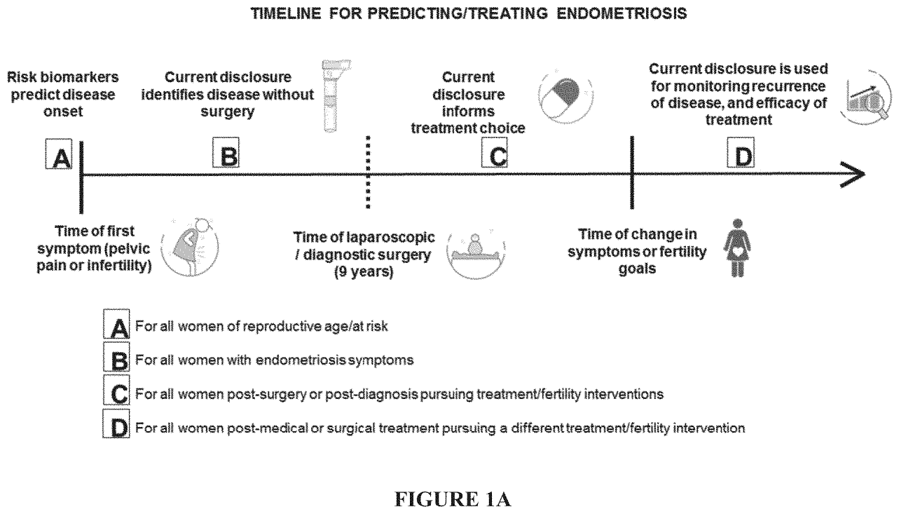

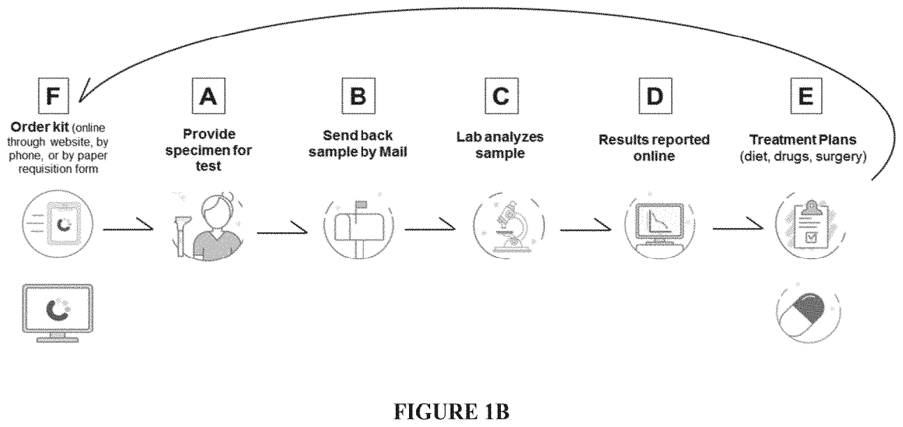

Referring to FIG. 1A, the methods and compositions herein may provide improvements to the traditional timeline of endometriosis diagnosis and treatment. The rapid, patient-centered diagnostic methods herein, which involve identification of endometriosis from easily collected samples can provide early prediction of being at risk of endometriosis (A), via a sample taken e.g. during a routine visit to an obstetrician-gynecologist (OB-GYN) or other medical professional or caregiver. Such methods may be especially useful for women of any reproductive age or with any endometriosis risk profile. Alternatively or additionally, the methods can provide generally quick diagnosis or detection of endometriosis following first onset of symptoms (B), which may avoid the average 7-10-year delay in typical detection of endometriosis via conventional laparoscopic procedures. As such, the methods and compositions provided herein may be especially useful for women with symptoms of endometriosis. In some embodiments, such detection or diagnosis of endometriosis may occur without surgery, such as laparoscopic surgery. Further, the methods or compositions herein may provide rapid or routine monitoring or detection of endometriosis severity (either in women diagnosed via the methods herein, or diagnosed via conventional laparoscopic procedures) in a way that may inform the initiation or adjustment of treatment procedures (C). As such, the methods and compositions provided herein are also especially useful for women who have previously had surgery, or have previously been diagnosed with endometriosis. In some cases, the women may be pursuing treatment or fertility interventions. In some cases, the methods or compositions provided herein may also provide monitoring of treatment efficacy, recurrence of disease, or disease severity on an ongoing basis (D). In some cases, monitoring of treatment efficacy, disease recurrence, or disease severity may occur after the subject has experienced a change in symptoms or fertility goals. In some cases, the methods and compositions provided herein may be useful for women after they have received medical or surgical treatment, particularly women who may be pursuing a different treatment or fertility intervention.

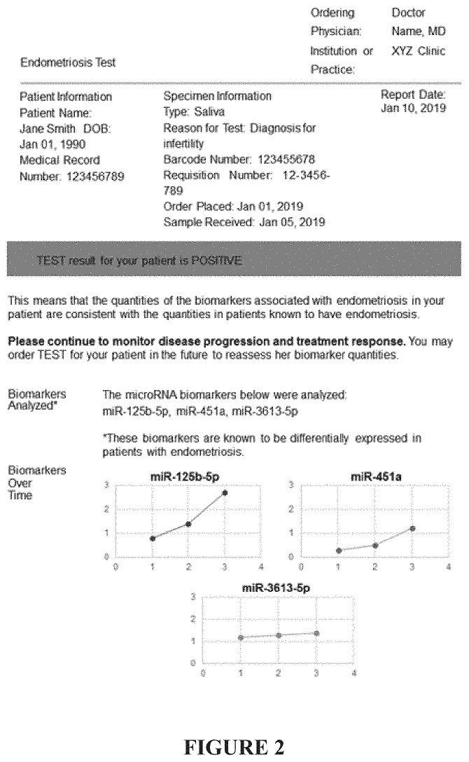

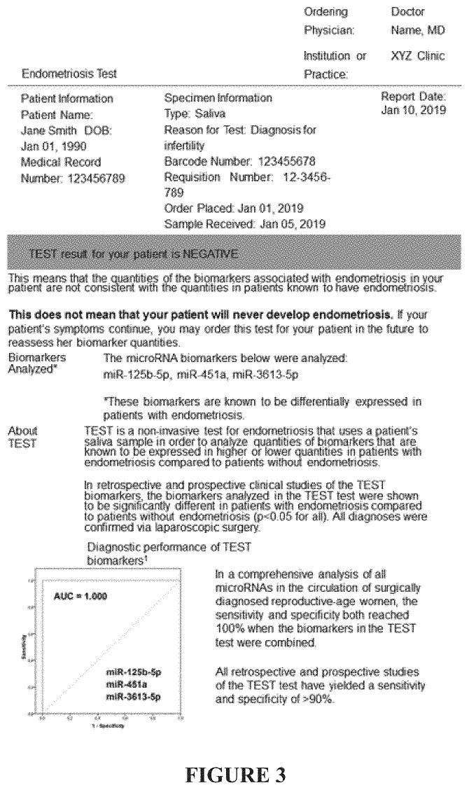

The testing and monitoring described herein may be provided via methods illustrated in FIG. 1B. A sample collection kit comprising the apparatuses and compositions for collection of a non-invasive sample may be ordered by the patient or patient's medical professional (such a kit may include e.g. an Oragene device for collection of saliva, or sample collection vials/stabilizing solutions suitable for collection of menstrual effluent or blood), F). The kit may be ordered, for example, online through a website, by phone, or by a paper requisition form. In some instances, the sample is collected or provided (A), and returned to the testing laboratory via the mail (B), after which the sample is examined for levels of one or more non-coding RNAs (e.g., miRNAs) associated with endometriosis (C). The expression data is used, in some cases, in combination with a machine learning model, to calculate a likelihood of endometriosis, which may be reported to the patient/caregiver (e.g. family member, home health aide) and/or patient's medical care provider online (D). In some cases, the results are reported online. Exemplary formats for such reports are provided in FIG. 2 and FIG. 3. The likelihood information may be used to inform treatment plans or future testing (e.g., laparoscopic procedures) or treatment with various agents used for treatment of endometriosis (e.g. progestins, GnRH agonists, GnRH antagonists, E). In some cases, the treatment plans may include a plan for the subject's diet or for surgical intervention. The entire process may be repeated on a regular basis (e.g., every two weeks, every month, every two months, every three months, every four months, every five months, every six months, every nine months, every year, every other year, every three years, etc.) to assess the ongoing severity of endometriosis and/or assess the effectiveness of the given treatment drug or dose of the drug.

In some cases, a patient is administered a treatment such as a GnRH agonist or antagonist (e.g., Elagolix). The non-coding RNA levels (e.g., miRNA levels) of the patient or subject may then be monitored over time. In some cases, the ncRNA levels (e.g., miRNA levels) are monitored or detected in a sample from the patient or subject over time. Such samples may include blood samples, menstrual blood samples, menstrual effluent samples, saliva samples, biopsy samples, samples comprising endometrial tissue, blood plasma samples, blood serum samples, urine samples, or any other biological samples. If the non-coding RNA (e.g., miRNA) profiles continue to indicate the presence of endometriosis, the GnRH agonist or antagonist (e.g., Elagolix) may be continued or increased. If the non-coding RNA (e.g., miRNA) profiles begin to indicate improvement of endometriosis, then the administration of the GnRH agonist or antagonist (e.g., Elagolix, a GnRH antagonist) may be reduced or eliminated.

Also provided herein are methods of detecting, diagnosing, monitoring, or treating endometriosis by detecting the levels of certain non-coding RNAs (ncRNA) (e.g., miRNAs) in menstrual effluent or menstrual blood. For example, a sample comprising menstrual blood or menstrual effluent may be obtained from a female subject, or provided by such female subject or her medical care provider. The sample comprising menstrual effluent or menstrual blood may be analyzed for levels of certain ncRNA (e.g. miRNA), including miRNA described further herein. Following detection of such ncRNA (e.g., miRNA) in the menstrual effluent or menstrual blood, the patient may be diagnosed with endometriosis, or be determined to be at risk of having endometriosis.

### Definitions

As used herein, the term “cell-free” refers to the condition of the nucleic acid as it appeared in the body directly before the sample is obtained from the body. For example, nucleic acids may be present in a body fluid such as blood or saliva in a cell-free state in that they are not associated with a cell. However, the cell-free nucleic acids may have originally been associated with a cell, such as an endometrial cell prior to entering the bloodstream or other body fluid. In contrast, nucleic acids that are solely associated with cells in the body are generally not considered to be “cell-free.” For example, nucleic acids extracted from a cellular sample are generally not considered “cell-free” as the term is used herein.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The terms “subject,” “patient,” “individual,” and the like are used interchangeably herein, and refer to any animal, or cells thereof whether in vitro or in situ, amenable to the methods described herein. In certain non-limiting embodiments, the patient, subject or individual is a human.

As used herein, “microRNA” or “miRNA” describes small non-coding RNA molecules, generally about 15 to about 50 nucleotides in length, preferably 17-23 nucleotides, which can play a role in regulating gene expression through, for example, a process termed RNA interference (RNAi). RNAi describes a phenomenon whereby the presence of an RNA sequence that is complementary or antisense to a sequence in a target gene messenger RNA (mRNA) results in inhibition of expression of the target gene. miRNAs are processed from hairpin precursors of about 70 or more nucleotides (pre-miRNA) which are derived from primary transcripts (pri-miRNA) through sequential cleavage by RNAse III enzymes. miRBase is a comprehensive microRNA database located at www.mirbase.org. In general, miRNA genes are transcribed into a precursor or pre miRNA that is processed into mature miRNA. pre-miRNA generally occurs in a hairpin form, wherein the hairpin contains a 5′ arm (or side) connected to a loop that is then connected to a 3′ arm (or side). Processing of the precursor miRNA can result in the formation of two mature forms of miRNA, including a 5p form that is derived from the 5′ side or arm of the precursor miRNA loop and a 3p form that is derived from the 3′ side or arm of the precursor miRNA hairpin.

As used herein, “let-7” may refer to let-7a, let-7b, let-7c, let-7d, let-7e, let-7f, and/or let-7g, in any combination.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

As used herein, the term “a” may be refer to a singular of plural form, in other word, “a” generally refers to “one or more.”

As used herein, RNA and RNAs are used interchangeably and may refer to a singular RNA or multiple RNA.

As used herein, “non-coding RNA” (ncRNA) generally refers to an endogenous RNA molecule that is not translated into a protein in a cell. Exemplary types of ncRNAs include transfer RNAs (tRNAs), ribosomal RNAs (rRNAs), microRNAs (miRNAs), piRNAs, snoRNAs, snRNAs, exRNAs, scRNAs and long ncRNAs (such as Xist and HOTAIR). In some embodiments, testing for endometriosis as described herein may involve determining the level of one or more ncRNA that is not a miRNA in addition to the specific microRNAs described herein.

Subjects

The methods and compositions described herein are applicable to human and non-human subjects, including veterinary subjects. Preferred subjects are “patients”—living humans that are receiving medical care for a disease or condition (e.g., endometriosis), or who are suspected of having such disease or condition or who are at risk of having such disease or condition. This includes persons with no defined illness who are being investigated for signs of pathology (e.g. endometriosis).

Preferred patients or subjects for the methods and compositions described herein are female patients that are at pubescent or post-pubescent ages, pre-menopausal, menopausal, or post-menopausal (as endometriosis may persist after menopause). As such, in general, the methods and compositions provided herein may be useful for female subjects within a large range of ages, generally over the age of 10. In some cases, a subject may be at risk of having endometriosis. Subject at risk of having endometriosis, may, for example, have a family history of endometriosis or a past medical history of endometriosis. In some cases, a subject may be suspected of having endometriosis. Such a subject may display no symptoms of endometriosis. But in other case, such subject may display symptoms of endometriosis such as dysmennorhea, pain with bowel movements or urination, infertility, or excessive bleeding.

Often, the subject is a patient or other individual undergoing a treatment regimen, or being evaluated for a treatment regimen (e.g., progestin therapy, GnRH agonist therapy, GnRH antagonist therapy). However, in some instances, the subject is not undergoing a treatment regimen. In some cases, the subject is receiving some kind of other medication, such as a pain reliever (e.g., non-steroidal, anti-inflammatory drug, NSAID). In some embodiments, the testing of one or more miRNA and/or ncRNA as described herein may provide the motivation for performing of assisted reproductive therapy (ART, e.g. in-vitro fertilization, IVF, or intrauterine insemination, IUI) such as in cases when endometriosis is detected.

In some instances the subject can have one or more symptoms associated with endometriosis. Such symptoms may include dysmennorhea, pain with bowel movements or urination, infertility or excessive bleeding. In some instances, the subject can have refractory endometriosis, which is the persistence of symptoms associated with endometriosis despite treatment with progestin (e.g. with oral birth control pills, dydrogesterone, medroxyprogesterone acetate, depot medroxyprogesterone acetate, or norethisterone).

Samples

The sample is preferably a bodily fluid sample. The bodily fluid may be sweat, saliva, tears, urine, blood, plasma, serum, vaginal fluid, cervico-vaginal fluid, whole blood, menstrual effluent (e.g. menstrual blood), spinal fluid, pulmonary fluid, sputum, or any other bodily fluid. In preferred embodiments, the sample is a saliva or menstrual effluent (e.g., menstrual blood) sample. In some cases, the sample comprises white blood cells (WBCs). In some cases, the sample comprises peripheral blood mononuclear cells (PBMCs); in some cases, the sample comprises peripheral blood lymphocytes (PBLs). As used herein, the term “saliva” does not include sputum, since sputum pertains to mucus or phlegm samples. In some embodiments, the saliva or menstrual effluent (e.g., blood) may be separated into cellular and non-cellular fractions by suitable methods (e.g., centrifugation, filtration). In some embodiments, nucleic acids (e.g., miRNA or ncRNA) may be extracted from the cellular (e.g. cell-containing) or non-cellular (e.g. non cell-containing) fractions. In some embodiments, analysis as described herein of miRNA or ncRNA expression may be performed on the cell-containing or non-cellular fractions of any of the samples (e.g. blood, plasma, serum, saliva, menstrual blood, menstrual effluent, etc.).

In some cases, the sample comprises tissue, such as tissue from a biopsy. In some cases, the tissue is endometrial tissue.

In some embodiments, the sample comprises cell-free non-coding RNA (e.g., cell-free miRNA). In some cases, the sample comprised purified or extracted non-coding RNA (e.g., miRNA). In some embodiments, the sample comprises exosome-encapsulated non-coding RNA (e.g., miRNA). In some embodiments, the sample comprises cell-encapsulated (e.g. by white blood cells) non-coding RNA (e.g., miRNA).

Sample Collection

As used herein “obtaining a sample” includes obtaining a sample directly or indirectly, including having a sample obtained (e.g., from a third party who directly obtained the sample from the subject). In some embodiments, the sample is taken from the subject by the same party (e.g. a testing laboratory) that subsequently acquires biomarker data from the sample. In some embodiments, the sample is received (e.g. by a testing laboratory) from another entity that collected it from the subject (e.g. a physician, nurse, phlebotomist, or other medical care provider). In some embodiments, the sample is taken from the subject by a medical professional under direction of a separate entity (e.g. a testing laboratory) and subsequently provided to said entity (e.g. the testing laboratory). In some embodiments, the sample is taken by the subject or the subject's caregiver (e.g. family member, home health aide) at home and subsequently provided to the party that acquires biomarker data from the sample (e.g. a testing laboratory).

In some embodiments, test samples of saliva may be obtained from a subject. Methods of obtaining saliva samples may include, but are not limited to ejection from the subject's mouth (e.g., spitting), aspiration, or removal by a swab or other collection tool. Methods for extracting RNA molecules from saliva can be found in e.g, Pandit, P et al. Clin Chem. 2013 July; 59(7):1118-22. A wide variety of saliva collection and recovery devices (which collect the sample in a clean manner and provide for the stabilization of nucleic acids in the sample) are available as kits and available from commercial providers such as DNA Genotek (e.g. Oragene-RNA and products described in US20110212002A1 and WO2008040126A1) and Norgen Biotek, and are suitable for use with the methods of the disclosure. Such kits are suitable for use by patients individually or with minimal assistance from a medical care provider (e.g. physician).

In some embodiments, test samples of menstrual effluent or menstrual blood may be obtained from a subject. Methods of obtaining menstrual effluent or menstrual blood samples include syringe aspiration (optionally in combination with use of a speculum), via collection with a menstrual cup, via collection from a tampon or menstrual pad, or other methods known in the art.

After collection, the menstrual effluent, menstrual fluid, or saliva samples may be stabilized by the addition of antimicrobial agents (e.g. Normocin, sodium azide), RNase inhibitors (e.g. Polyvinylsulfonic acid, RNasin®, RNaseOUT™), by disruption in organic solution (e.g. Trizol, phenol-chloroform, phenol-chloroform-isoamyl alcohol), or by disruption in detergents in combination with broad-spectrum proteases (e.g. SDS with Proteinase K).

In some embodiments, test samples of blood may be obtained from a subject. In some embodiments, the blood sample is a peripheral blood sample. In some embodiments, the blood sample is a whole blood sample. In some embodiments, the sample is a blood sample and comprises whole blood, peripheral blood, serum, plasma, PBLs, PBMCs, T cells, CD4 T cells CD8 T cells, or macrophages. The blood sample may be obtained by a minimally-invasive method such as a blood draw. The blood sample may be obtained by venipuncture.

RNA (e.g., miRNA) Expression Profiling.

The methods, kits, and systems disclosed herein may comprise specifically detecting, profiling, or quantitating RNAs (e.g., ncRNAs, miRNAs) that are within the biological samples to determine an expression profile. In some instances, RNAs (e.g. miRNAs, ncRNAs) may be isolated from the biological samples. In some cases, RNAs (e.g. miRNAs, ncRNAs) may be isolated from a cell-free source.

Expression profiles are generally measured by detecting levels of cDNA derived from miRNA or other type of ncRNA. Expression profiles may also be measured at the RNA level; e.g. by RNA hybridization or direct RNA sequencing.

In some cases, expression levels are determined by so-called “real time amplification” methods also known as quantitative PCR (qPCR) or Taqman. The basis for this method of monitoring the formation of amplification product formed during a PCR reaction with a template using oligonucleotide probes/oligos specific for a region of the template to be detected. In some embodiments, qPCR or Taqman are used immediately following a reverse-transcriptase reaction performed on isolated RNAs (e.g. miRNAs, ncRNAs) and can be used to quantitate the levels of the RNA, and/or assess the differential expression levels of the RNAs (e.g. miRNAs, ncRNAs.

Taqman uses a dual-labeled fluorogenic oligonucleotide probe. The dual labeled fluorogenic probe used in such assays is typically a short (ca. 20-25 bases) polynucleotide that is labeled with two different fluorescent dyes. The 5′ terminus of the probe is typically attached to a reporter dye and the 3′ terminus is attached to a quenching dye. Regardless of labelling or not, the qPCR probe is designed to have at least substantial sequence complementarity with a site on the target RNA (e.g. miRNA, ncRNA) or nucleic acid derived from. Upstream and downstream PCR primers that bind to flanking regions of the locus are also added to the reaction mixture. When the probe is intact, energy transfer between the two fluorophores occurs and the quencher quenches emission from the reporter. During the extension phase of PCR, the probe is cleaved by the 5′ nuclease activity of a nucleic acid polymerase such as Taq polymerase, thereby releasing the reporter from the polynucleotide-quencher and resulting in an increase of reporter emission intensity which can be measured by an appropriate detector. The recorded values can then be used to calculate the increase in normalized reporter emission intensity on a continuous basis and ultimately quantify the amount of the RNA (e.g. miRNA, ncRNA) being amplified. RNA (e.g. miRNA, ncRNA) levels can also be measured without amplification by hybridization to a probe, for example, using a branched nucleic acid probe, such as a QuantiGene® Reagent System from Panomics. This format of test is particularly useful for the multiplex detection of multiple genes/miRNAs from a single sample reaction, as each fluorophore/quencher pair attached to an individual probe may be spectrally orthogonal to the other probes used in the reaction such that multiple probes (each directed against a different miRNA/gene product or other ncRNA gene product) can be detected during the amplification/detection reaction.

qPCR can also be performed without a dual-labeled fluorogenic probe by using a fluorescent dye (e.g. SYBR Green) that specifically intercalates into dsDNA and reflects the accumulation of dsDNA amplified specific upstream and downstream oligonucleotide primers. The increase in fluorescence during the amplification reaction is followed on a continuous basis and can be used to quantify the amount of RNAs (e.g. miRNAs or other ncRNAs) being amplified.

For qPCR or Taqman, the levels of particular miRNA or ncRNA genes may be expressed relative to one or more internal control RNAs (e.g., miRNA, ncRNA) measured from the same sample using the same detection methodology. Internal control RNA (e.g., miRNA, ncRNA) may include so-called constitutively expressed RNA such as U6, RNU48, RNU44, U47, RNU6B, or a combination thereof.

In some embodiments, for qPCR or Taqman detection, a “pre-amplification” step is performed on cDNA transcribed from RNA (e.g. miRNA, ncRNA) prior to the quantitatively monitored PCR reaction. This serves to increase signal in conditions where the natural level of the RNA/cDNA to be detected is very low. Suitable methods for pre-amplification include but are not limited LM-PCR, and PCR with random oligonucleotide primers (e.g. random hexamer PCR), and any combination thereof.

In some embodiments, for qPCR or Taqman detection, an RT-PCR step is first performed to generate cDNA from RNA (e.g. miRNA, ncRNA). Such amplification by RT-PCR can either be general (e.g. amplification with partially/fully degenerate oligonucleotide primers) or targeted (e.g. amplification with oligonucleotide primers directed against specific RNAs (e.g., miRNA, ncRNA) which are to be analyzed at a later step).

In other methods, expression levels are determined by sequencing, such as by RNA sequencing or by DNA sequencing (e.g., of cDNA generated from reverse-transcribing RNA, ncRNA or miRNA from a sample). Sequencing may also be general (e.g. with amplification using partially/fully degenerate oligonucleotide primers) or targeted (e.g. with amplification using oligonucleotide primers directed against specific RNAs (e.g. miRNA, ncRNA) that are to be analyzed at a later step). Sequencing may be performed by any available method or technique. Sequencing methods may include: Next Generation sequencing, high-throughput sequencing, pyrosequencing, classic Sanger sequencing methods, sequencing-by-ligation, sequencing by synthesis, sequencing-by-hybridization, RNA-Seq (Illumina), Digital Gene Expression (Helicos), next generation sequencing, single molecule sequencing by synthesis (SMSS) (Helicos), Ion Torrent Sequencing Machine (Life Technologies/Thermo-Fisher), massively-parallel sequencing, clonal single molecule Array (Solexa), shotgun sequencing, nanopore sequencing (e.g. Oxford Nanopore Technologies platforms), Maxim-Gilbert sequencing, or primer walking.

In other methods, expression levels are determined by a hybridization-based method, such as Northern blot, Southern blot, or microarray hybridization.

Biomarker RNAs (e.g., miRNA, ncRNA).

The methods and compositions herein may involve the detection of at least one ncRNA (e.g., miRNA) associated with endometriosis (e.g., detection of presence or absence of the at least on ncRNA) or measurement of a level of at least one miRNA or ncRNA associated with endometriosis from a patient sample to detect, predict, or monitor the severity of endometriosis. Such markers may include Let-7a, Let-7b, Let-7c, Let-7d, Let-7e, Let-7f, miR-135a, miR-135b, miR-18a, miR-125b, miR-143, miR-145, miR-150, miR-342, miR-451a, miR-500a, miR-3613, and miR-6755 individually or in any combination. Such markers may include let-7a, let-7b, let-7c, let-7d, let-7e, let-7f, miR-135a, miR-135b, miR-18a, miR-125b, miR-143, miR-145, miR-150, miR-342, miR-451a, miR-500a, miR-3613, and miR-6755 individually or in any combination. In some embodiments, the methods and compositions involve detection of the −3p or −5p transcript of these miRNAs. In some embodiments, the methods or compositions herein involve the testing of levels of one or more miRNAs from particular sets of miRNAs. In some embodiments, the one or more miRNA is selected from the group consisting of let-7c, let-7d, let-7f, miR-18a, miR-125b, miR-143, miR-150, miR-342, miR-451a, miR-500a, miR-3613, and miR-6755, or any combination thereof. In some embodiments, the one or more miRNA is selected from the group consisting of let-7a, let-7b, let-7c, let-7d, let-7e, let-7f, mir135a, and mir135b, or any combination thereof. In some embodiments, the one or more miRNA is selected from the group consisting of miR-125b, miR-150, miR-342, miR-451a, miR-3613, and let-7b, or any combination thereof. In some embodiments, the at least one miRNA is selected from the group consisting of miR-150, 451a, and 3613, or any combination thereof.

In some embodiments, the methods and compositions herein involve the detection or measurement of at least one ncRNA (e.g., miRNA) as outlined herein in combination with at least one ncRNA that is not an miRNA. Such ncRNAs include tRNAs, rRNAs, piRNAs, snoRNAs, snRNAs, exRNAs, scRNAs and long ncRNAs, or any combination thereof. In some embodiments, the methods and compositions involve measurement or detection of at least one ncRNA.

In some cases, the detected biomarker RNA (e.g., miRNA, ncRNA) is cell-free RNA (e.g., ncRNA, miRNA), particularly RNA that is obtained from a cell-free fraction of a sample. In some cases, the detected biomarker RNA (e.g., miRNA, ncRNA) are cell-associated RNA (e.g. cell-associated miRNA, cell-associated ncRNA). In some cases, the detected biomarker RNA (e.g., miRNA, ncRNA) are RNAs (e.g., miRNA, ncRNA) present in cells (or associated with cells) such as white blood cells or endometrial cells. In some cases, the detected biomarker RNAs (e.g., miRNA, ncRNA) are RNAs (e.g, miRNA, ncRNA) present in or associated with exosomes.

Analyzing the gene expression profile may comprise normalizing the RNA (e.g. miRNA, ncRNA) level from the subject. In some embodiments, the RNA is normalized relative to the determined expression level of a constitutive RNA such as small nuclear RNA U6, RNU48, RNU44, U47, or RNU6B, or any combination thereof.

Sample Classification

The methods provided herein may include using a trained classifier or algorithm to analyze sample data, particularly to detect endometriosis. In some instances, the levels of RNA (e.g, miRNA, ncRNA) from a sample are used to develop or train an algorithm or classifier provided herein. In some instances, RNA levels (e.g., miRNA, ncRNA levels) are measured in a sample from an asymptomatic patient or a patient having one or more symptom of endometriosis and a classifier or algorithm (e.g., trained algorithm) is applied to the resulting data in order to detect, predict, or monitor endometriosis.

Training of multi-dimensional classifiers (e.g., algorithms) may be performed using numerous samples. For example, training of the multi-dimensional classifier may be performed using at least about 10, 20, 30, 40, 50, 60, 70, 80, 90, 100, 110, 120, 130, 140, 150, 160, 170, 180, 190, 200 or more samples. In some cases, training of the multi-dimensional classifier may be performed using at least about 200, 210, 220, 230, 240, 250, 260, 270, 280, 290, 300, 350, 400, 450, 500 or more samples. In some cases, training of the multi-dimensional classifier may be performed using at least about 525, 550, 600, 650, 700, 750, 800, 850, 900, 950, 1000, 1100, 1200, 1300, 1400, 1500, 1600, 1700, 1800, 2000 or more samples.

Further disclosed herein are classifier sets and methods of producing one or more classifier sets. The classifier set may comprise one or more RNAs (e.g. miRNAs, ncRNA), such as let-7a, let-7b, let-7c, let-7d, let-7e, let-7f, miR-135a, miR-135b, miR-18a, miR-125b, miR-143, miR-145, miR-150, miR-342, miR-451a, miR-500a, miR-3613, and miR-6755 individually or in any combination. Disclosed herein is the use of a classification system comprises one or more classifiers.

Classifiers and/or classifier probe sets may be used to either rule-in or rule-out a sample as healthy. For example, a classifier may be used to classify a sample as being from a healthy subject. Alternatively, a classifier may be used to classify a sample as being from an unhealthy subject. Alternatively, or additionally, classifiers may be used to either rule-in or rule-out a sample as endometriosis. For example, a classifier may be used to classify a sample as being from a subject suffering from endometriosis. In another example, a classifier may be used to classify a sample as being from a subject that is not suffering from endometriosis.

The methods disclosed herein may comprise assigning a classification to one or more samples from one or more subjects. Assigning the classification to the sample may comprise applying an algorithm to the level of one or more RNA (e.g. miRNA, ncRNA) from the sample.

The algorithm may provide a record of its output including a classification of a sample and/or a confidence level. In some instances, the output of the algorithm can be the possibility of the subject of having a condition, such as endometriosis.

The algorithm may be a trained algorithm. The algorithm may comprise a linear classifier. The linear classifier may comprise one or more linear discriminant analysis, Fisher's linear discriminant, Naïve Bayes classifier, Logistic regression, Perceptron, Support vector machine, or a combination thereof. The linear classifier may be a Support vector machine (SVM) algorithm.

The algorithm may comprise one or more linear discriminant analysis (LDA), Basic perceptron, Elastic Net logistic regression, logistic regression, (Kernel) Support Vector Machines (SVM), Diagonal Linear Discriminant Analysis (DLDA), Golub Classifier, Parzen-based, (kernel) Fisher Discriminant Classifier, k-nearest neighbor, Iterative RELIEF, Classification Tree, Maximum Likelihood Classifier, Random Forest, Nearest Centroid, Prediction Analysis of Microarrays (PAM), k-medians clustering, Fuzzy C-Means Clustering, Gaussian mixture models, or a combination thereof. The algorithm may comprise a Diagonal Linear Discriminant Analysis (DLDA) algorithm. The algorithm may comprise a Nearest Centroid algorithm. The algorithm may comprise a Random Forest algorithm.

The methods provided herein can help determine whether the patient has endometriosis with a high degree of accuracy, sensitivity, and/or specificity. In some cases, the predictive accuracy (e.g., for detecting endometriosis, or for distinguishing endometriosis from non-endometriosis) is greater than 75%, 85%, 90%, 91%, 92%, 93%, 94%, 95%, 96%, 97%, 98%, 98.5%, 99.0%, 99.1%, 99.2%, 99.3%, 99.4%, 99.5%, 99.6%, 99.7%, 99.8%, 99.9%, 99.95%, or 99.99%. In some embodiments, the predictive accuracy is 100%. In some cases, the sensitivity (e.g., for detecting endometriosis, or for distinguishing endometriosis from non-endometriosis) is greater than 75%, 85%, 90%, 91%, 92%, 93%, 94%, 95%, 96%, 97%, 98%, 98.5%, 99.0%, 99.1%, 99.2%, 99.3%, 99.4%, 99.5%, 99.6%, 99.7%, 99.8%, 99.9%, 99.95%, or 99.99%. In some embodiments the sensitivity is 100%. In some cases, the specificity (e.g., for detecting endometriosis, or for distinguishing endometriosis from non-endometriosis) is greater than 75%, 85%, 90%, 91%, 92%, 93%, 94%, 95%, 96%, 97%, 98%, 98.5%, 99.0%, 99.1%, 99.2%, 99.3%, 99.4%, 99.5%, 99.6%, 99.7%, 99.8%, 99.9%, 99.95%, or 99.99%. In some cases, the specificity is 100%. In some cases, the positive predictive value (e.g., for detecting endometriosis, or for distinguishing endometriosis from non-endometriosis) of the method is greater than 75%, 85%, 90%, 91%, 92%, 93%, 94%, 95%, 96%, 97%, 98%, 98.5%, 99.0%, 99.1%, 99.2%, 99.3%, 99.4%, 99.5%, 99.6%, 99.7%, 99.8%, 99.9%, 99.95%, or 99.99%. In some cases the positive predictive value is 100%. The AUC after thresholding in any of the methods provided herein may be greater than 0.9, 0.91, 0.92, 0.93, 0.94, 0.95, 0.96, 0.97, 0.98, 0.99, 0.995, or 0.999. Conversely, the method may predict or determine whether a subject does not have or is at reduced risk of endometriosis. The negative predictive value (e e.g., for detecting endometriosis, or for distinguishing endometriosis from non-endometriosis) may be greater than 85%, 90%, 91%, 92%, 93%, 94%, 95%, 96%, 97%, 98%, 98.5%, 99.0%, 99.1%, 99.2%, 99.3%, 99.4%, 99.5%, 99.6%, 99.7%, 99.8%, 99.9%, 99.95%, or 99.99%. In some cases, the negative predictive value is 100%.

Computer-Implemented Methods

Expression levels of one or more RNAs (e.g. miRNA, ncRNA) can be analyzed and associated with status of a subject (e.g., endometriosis) in a digital computer. Optionally, such a computer is directly linked to a scanner or the like (e.g. a qPCR system, a multiplex fluorescent plate reader, FACS instrument, or a sequencer) receiving experimentally determined signals related to miRNA or ncRNA expression levels. Alternatively, expression levels can be input by other means. The computer can be programmed to convert raw signals into expression levels (absolute or relative), compare measured expression levels with one or more reference expression levels, or a scale of such values, as described above. The computer can also be programmed to assign values or other designations to expression levels based on the comparison with one or more reference expression levels, and to aggregate such values or designations for multiple genes in an expression profile. The computer can also be programmed to output a value or other designation providing an indication of presence of endometriosis as well as any of the raw or intermediate data used in determining such a value or designation.

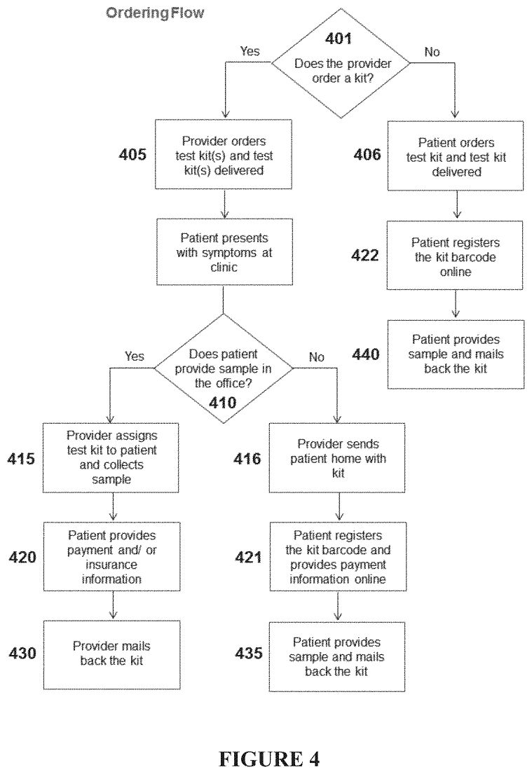

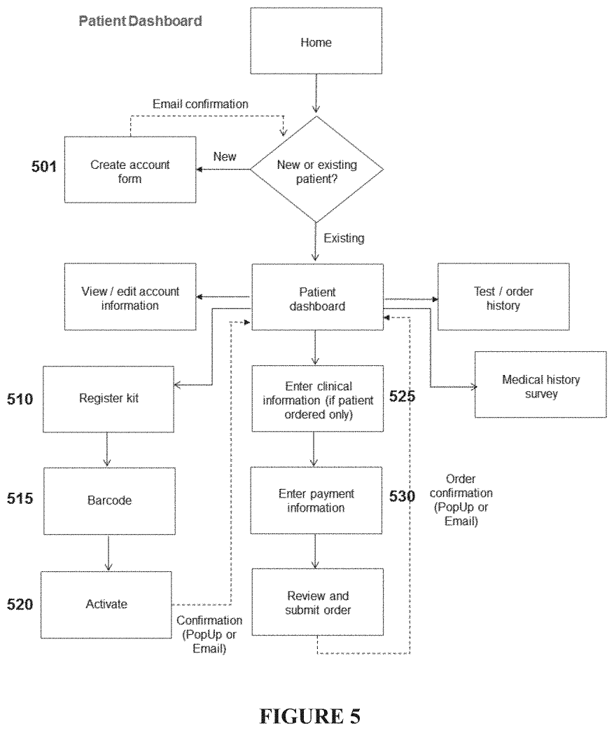

A typical computer (see U.S. Pat. No. 6,785,613 FIGS. 4 and 5) may include a bus which interconnects major subsystems such as a central processor, a system memory, an input/output controller, an external device such as a printer via a parallel port, a display screen via a display adapter, a serial port, a keyboard, a fixed disk drive and a floppy disk drive operative to receive a floppy disk. Many other devices can be connected such as a scanner via I/O controller, a mouse connected to serial port or a network interface. The computer contains computer readable media holding codes to allow the computer to perform a variety of functions. These functions include controlling automated apparatus, receiving input and delivering output as described above. The automated apparatus can include a robotic arm for delivering reagents for determining expression levels, as well as small vessels, e.g., microtiter wells for performing the expression analysis.

The methods, systems, kits and compositions provided herein may also be capable of generating and transmitting results through a computer network. In some cases, a sample is first collected from a subject (e.g. a patient with one or more symptoms of endometriosis, or a non-symptomatic patient). In some instances, the sample is assayed and RNA (e.g. miRNA, ncRNA) levels are measured. A computer system may be used in analyzing the data and making classification of the sample. The result may be capable of being transmitted to different types of end users via a computer network. In some instances, the subject (e.g. patient) may be able to access the result by using standalone software and/or a web-based application on a local computer capable of accessing the internet. In some instances, the result can be accessed via a mobile application provided to a mobile digital processing device (e.g. mobile phone, tablet, etc.). In some instances, the result may be accessed by medical care provider (e.g. physician) s and help them identify and track conditions of their patients. In some instances, the result may be used for other purposes such as education and research.

Computer Program

The methods, kits, and systems disclosed herein may include at least one computer program, or use of the same. A computer program may include a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. The computer program will normally provide a sequence of instructions from one location or a plurality of locations. In various embodiments, a computer program includes, in part or in whole, one or more web applications, one or more mobile applications, one or more standalone applications, one or more web browser plug-ins, extensions, add-ins, or add-ons, or combinations thereof.

Further disclosed herein are systems for classifying one or more samples and uses thereof. The system may comprise (a) a digital processing device comprising an operating system configured to perform executable instructions and a memory device; (b) a computer program including instructions executable by the digital processing device to classify a sample from a subject comprising: (i) a first software module configured to receive a RNA (e.g. miRNA, ncRNA) expression profile of one or more RNA (e.g. miRNA, ncRNA) from the sample from the subject; (ii) a second software module configured to analyze the RNA (e.g. miRNA, ncRNA) expression profile from the subject; and (iii) a third software module configured to classify the sample from the subject based on a classification system comprising two or more classes. At least one of the classes may be selected from endometriosis. Analyzing the gene expression profile from the subject may comprise applying an algorithm. Analyzing the gene expression profile may comprise normalizing the RNA (e.g. miRNA, ncRNA) expression profile from the subject (e.g. to a constitutive RNA such as small nuclear RNA U6, RNU48, RNU44, U47, or RNU6B, or any combination thereof.

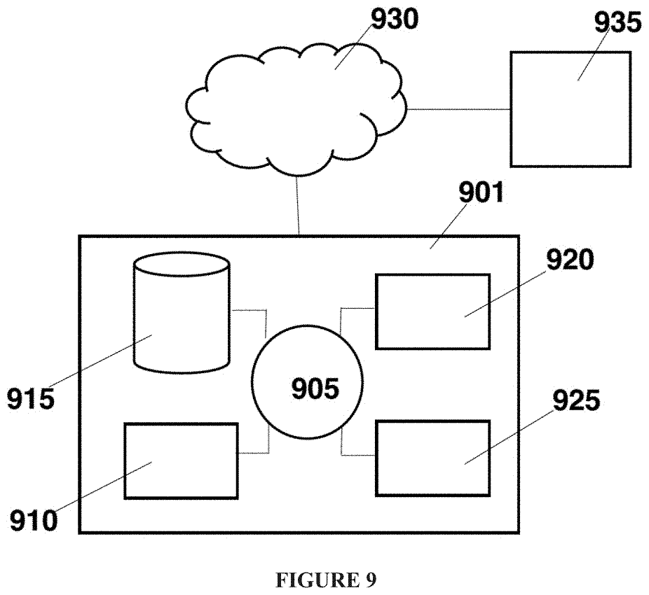

FIG. 9 shows a computer system (also “system” herein) 901 programmed or otherwise configured for implementing the methods of the disclosure, such as producing a selector set and/or for data analysis. The system 901 includes a central processing unit (CPU, also “processor” and “computer processor” herein) 905, which can be a single core or multi core processor, or a plurality of processors for parallel processing. The system 901 also includes memory 910 (e.g., random-access memory, read-only memory, flash memory), electronic storage unit 915 (e.g., hard disk), communications interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communications bus (solid lines), such as a motherboard. The storage unit 915 can be a data storage unit (or data repository) for storing data. The system 901 is operatively coupled to a computer network (“network”) 930 with the aid of the communications interface 920. The network 930 can be the Internet, an internet and/or extranet, or an intranet and/or extranet that is in communication with the Internet. The network 930 in some instances is a telecommunication and/or data network. The network 930 can include one or more computer servers, which can enable distributed computing, such as cloud computing. The network 930 in some instances, with the aid of the system 901, can implement a peer-to-peer network, which may enable devices coupled to the system 901 to behave as a client or a server.

The system 901 is in communication with a processing system 935. The processing system 935 can be configured to implement the methods disclosed herein. In some examples, the processing system 935 is a multiplex fluorescent plate reader, a qPCR machine, or a nucleic acid sequencing system, such as, for example, a next generation sequencing system (e.g., Illumina sequencer, Ion Torrent sequencer, Pacific Biosciences sequencer). The processing system 935 can be in communication with the system 901 through the network 930, or by direct (e.g., wired, wireless) connection. The processing system 935 can be configured for analysis, such as nucleic acid sequence analysis.

Methods as described herein can be implemented by way of machine (or computer processor) executable code (or software) stored on an electronic storage location of the system 901, such as, for example, on the memory 910 or electronic storage unit 915. During use, the code can be executed by the processor 905. In some examples, the code can be retrieved from the storage unit 915 and stored on the memory 910 for ready access by the processor 905. In some situations, the electronic storage unit 915 can be precluded, and machine-executable instructions are stored on memory 910.

Digital Processing Device

The methods, kits, and systems disclosed herein may include a digital processing device, or use of the same. In further embodiments, the digital processing device includes one or more hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

The digital processing device will normally include an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

The device generally includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

A display to send visual information to a user will normally be initialized. Examples of displays include a cathode ray tube (CRT, a liquid crystal display (LCD), a thin film transistor liquid crystal display (TFT-LCD, an organic light emitting diode (OLED) display. In various further embodiments, on OLED display is a passive-matrix OLED (PMOLED) or active-matrix OLED (AMOLED) display. In some embodiments, the display may be a plasma display, a video projector or a combination of devices such as those disclosed herein.

The digital processing device would normally include an input device to receive information from a user. The input device may be, for example, a keyboard, a pointing device including, by way of non-limiting examples, a mouse, trackball, track pad, joystick, game controller, or stylus; a touch screen, or a multi-touch screen, a microphone to capture voice or other sound input, a video camera to capture motion or visual input or a combination of devices such as those disclosed herein.

Non-Transitory Computer Readable Storage Medium

The methods, kits, and systems disclosed herein may include one or more non-transitory computer readable storage media encoded with a program including instructions executable by the operating system to perform and analyze the test described herein; preferably connected to a networked digital processing device. The computer readable storage medium is a tangible component of a digital that is optionally removable from the digital processing device. The computer readable storage medium includes, by way of non-limiting examples, CD-ROMs, DVDs, flash memory devices, solid state memory, magnetic disk drives, magnetic tape drives, optical disk drives, cloud computing systems and services, and the like. In some instances, the program and instructions are permanently, substantially permanently, semi-permanently, or non-transitorily encoded on the media.

A non-transitory computer-readable storage media may be encoded with a computer program including instructions executable by a processor to create or use a classification system. The storage media may comprise (a) a database, in a computer memory, of one or more clinical features of two or more control samples, wherein (i) the two or more control samples may be from two or more subjects; and (ii) the two or more control samples may be differentially classified based on a classification system comprising three or more classes; (b) a first software module configured to compare the one or more clinical features of the two or more control samples; and (c) a second software module configured to produce a classifier set based on the comparison of the one or more clinical features.

At least two of the classes may be selected from endometriosis, non-endometriosis, and healthy.

Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

Web Browser Plug-in

In some embodiments, the computer program includes a web browser plug-in. In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

Software Modules

The methods, kits, and systems disclosed herein may include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

Databases

The methods, kits, and systems disclosed herein may comprise one or more databases, or use of the same. In view of the disclosure provided herein, those of skill in the art will recognize that many databases are suitable for storage and retrieval of information pertaining to miRNA or ncRNA expression profiles, sequencing data, classifiers, classification systems, therapeutic regimens, or a combination thereof. In various embodiments, suitable databases include, by way of non-limiting examples, relational databases, non-relational databases, object oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local computer storage devices.

Data Transmission

The methods, kits, and systems disclosed herein may be used to transmit one or more reports. The one or more reports may comprise information pertaining to the classification and/or identification of one or more samples from one or more subjects. The one or more reports may comprise information pertaining to a disease status (e.g. endometriosis or non-endometriosis). The one or more reports may comprise information pertaining to therapeutic regimens for use in treating endometriosis in a subject in need thereof. The one or more reports may be transmitted to a subject or a medical representative of the subject. The medical representative of the subject may be a physician, physician's assistant, nurse, or other medical care provider. The medical representative of the subject may be a family member of the subject. A family member of the subject may be a parent, guardian, child, sibling, aunt, uncle, cousin, or spouse. The medical representative of the subject may be a legal representative of the subject.

Methods of Testing Using RNA (e.g. miRNA, ncRNA) Sampling

In some aspects the present disclosure provides novel methods of conducting a test such as a diagnostic test and of reporting the results to the subject's medical care provider. In some cases, the human subject directly obtains the diagnostic kit, such as through a retail purchase at a drug store or internet commerce site; in some cases, a medical care provider (e.g. physician) care provider orders the diagnostic kit for the human subject. The human subject may have or be suspected of having a disease or disorder such as endometriosis.

The methods may involve (a) providing to the subject a saliva, menstrual blood, or menstrual effluent sampling kit, wherein the saliva, menstrual blood or menstrual effluent sampling kit comprises: (i) a saliva, menstrual blood or menstrual effluent recovery and collection device; and (ii) a code which uniquely identifies the saliva, menstrual blood or menstrual effluent recovery and collection device. The saliva, menstrual blood or menstrual effluent sampling kit may additionally comprise other components, such as packaging with pre-paid postage to protect the saliva, menstrual blood or menstrual effluent sample when it is sent in for testing, instructions for using the saliva, menstrual blood or menstrual effluent recovery and collection device, and instructions for registering the code which uniquely identifies the saliva, menstrual blood or menstrual effluent collection kit via a web based interface. An exemplary scheme for providing saliva, menstrual blood or menstrual effluent sampling kits is described in FIG. 4. The saliva, menstrual blood or menstrual effluent sampling kit may be provided to the subject via a variety of routes, depending on if the kit is ordered by the medical care provider or not (401): it may be mailed to the subject at their personal address (406) or mailed to the subject's medical care provider (e.g. physician) care provider at their place of business and provided to the subject during an appointment (405). In some embodiments the saliva, menstrual blood or menstrual effluent sampling kit may be provided out of a pool of kits previously mailed to the medical care provider (e.g. physician) at their place of business (415). In other embodiments the saliva, menstrual blood or menstrual effluent sampling kit may be separately mailed to the medical care provider and intended for the use by a particular patient. Likewise, the cost of the kit may be billed via a variety of routes. In one embodiment, the cost of the saliva, menstrual blood or menstrual effluent sampling kit and diagnostic testing is billed directly to the subject's credit card account or the subject's bank account (420 or 421). This transaction may be patient-initiated, with the patient providing their own credit card account or bank account number (421). The transactions performed with a credit card account or bank account may also be accomplished via cash or check transactions. This transaction may also be medical care provider-initiated, with the medical care provider (e.g. physician) providing the patient's credit card account number (420).

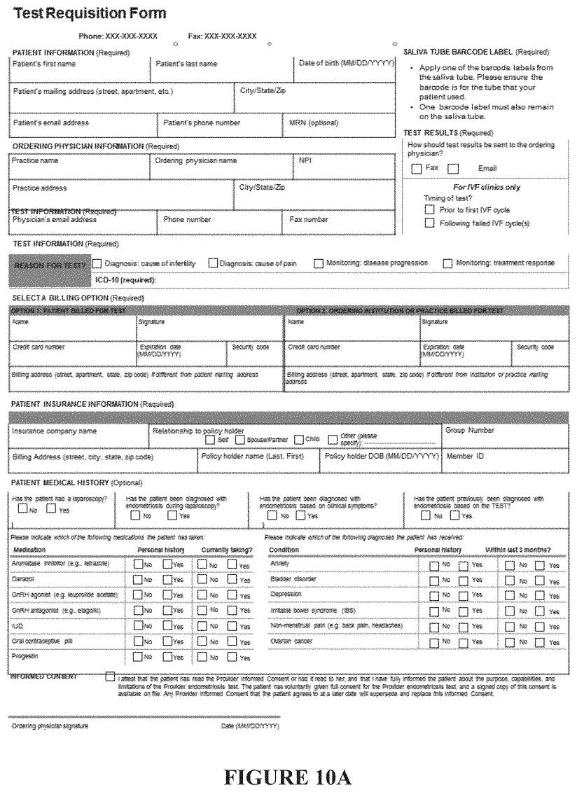

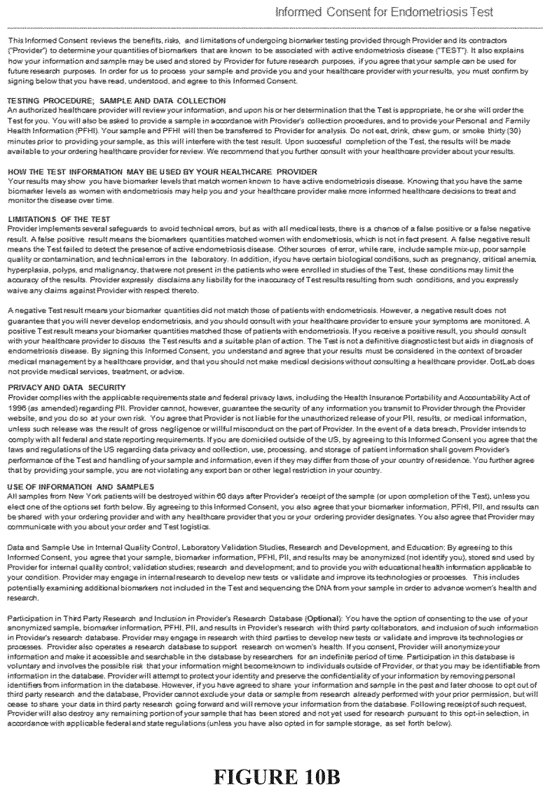

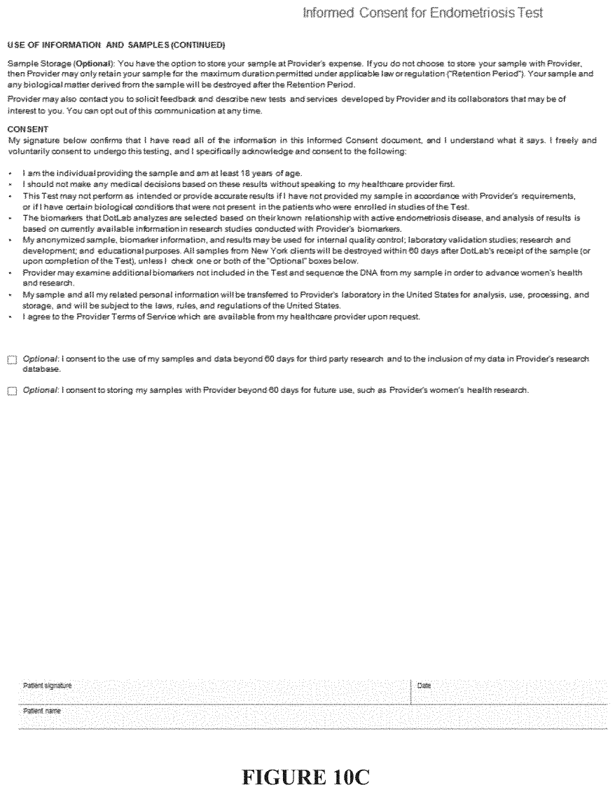

Alternatively or additionally, the test may be provided to the patient upon the filling of a (e.g. paper- or web-based) form. Exemplary forms are presented in FIG. 10A, FIG. 10B, and FIG. 10C. The forms may supply the same information as a web portal relevant to the provision and interpretation of the endometriosis test (see FIG. 10A), such as medical provider identification information, saliva (or menstrual blood/menstrual effluent) tube barcode label, patient billing information, diagnosis stage (e.g. confirmed with laparoscopy or not, exhibiting clinical symptoms), any ongoing medication (e.g. aromatase inhibitors, danazol, GnRH agonists, GnRH antagonists, oral contraceptives, progestins), and any related clinical conditions relevant for treatment (e.g. anxiety, bladder disorder, depression, irritable bowel syndrome, non-menstrual pain, ovarian cancer). The (e.g. paper- or web-based) form may also involve the provision and signing of informed consent information such as procedures for sample testing and data collection, interpretation, test limitations, privacy and data security, non-test usage and storing of samples (see FIG. 10B and FIG. 10C). The forms may be provided by either the patient's medical care provider or the patient.

The methods may next involve a series of actions to receive the saliva, menstrual blood or menstrual effluent sample and ensure proper handling of the sample such that it can be associated with the proper patient/medical care provider (e.g. patient/physician) combination.

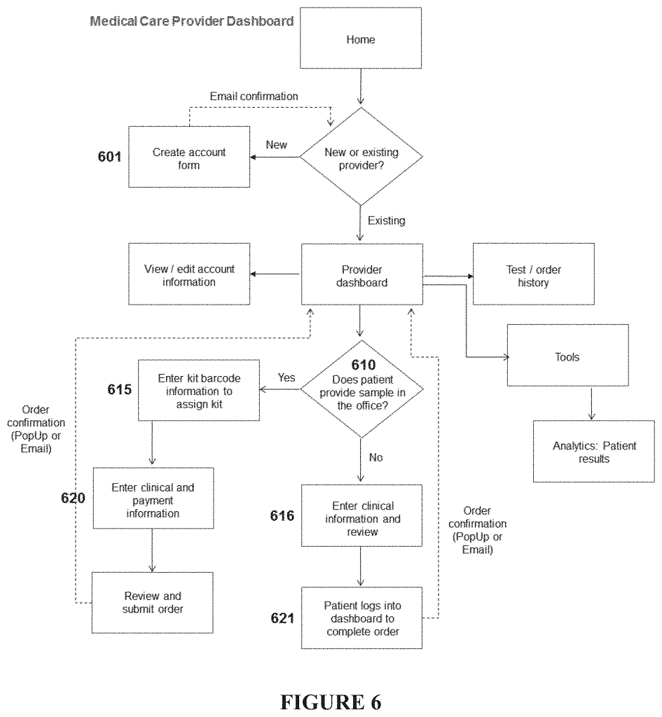

In some embodiments, the subject is first assigned, in a first database, a code which uniquely identifies the subject. This assigning may be in response to a patient creating a web account with the applicant (see e.g. FIG. 5, 501). Alternatively, the assigning is based on the medical care provider (e.g. physician) registering a saliva, menstrual blood or menstrual effluent sampling kit via their own web-based interface prior to submitting a saliva, menstrual blood or menstrual effluent sample (see e.g. FIG. 6, 601). In any event, exemplary flows for providing test-related information via a web portal for the patient and the physician/medical provider are outlined in FIG. 5 and FIG. 6.

Whatever route is chosen, two items may then be received separately from the subject: (i) a saliva sample in the saliva sampling kit (or menstrual effluent sample (or menstrual blood sample) in the menstrual effluent (or blood) sampling kit); (ii) the code which uniquely identifies the saliva sampling kit (or menstrual effluent (or blood) sampling kit); and (iii) a pre-assigned code which uniquely identifies the patient's medical care provider (e.g. physician) or medical care provider. The route of provision depends on if the patient provides the sample in the medical care provider's place of business (e.g. office) or not (410). In some embodiments, the saliva sample, menstrual blood or menstrual effluent sample is mailed from the patient's home address (or preferred mailing address) after the saliva sampling kit (or menstrual effluent sampling kit, or menstrual blood sampling kit) is received at the patient's home address (or preferred mailing address) and the saliva sample (or menstrual effluent sample) is collected without assistance by the patient at their home address or preferred mailing address (435 or 440). In other embodiments, the saliva sample (or menstrual effluent sample, or menstrual blood sample) is mailed from the patient's home address (or preferred mailing address) after the saliva sampling kit (or menstrual effluent (or blood) sampling kit) is received by the patient at the medical care provider's place of business and the saliva sample (or menstrual effluent sample, or menstrual blood sample) is collected without assistance by the patient at their home address (or preferred mailing address). In yet other embodiments, the saliva sample, menstrual blood sample, or menstrual effluent sample is mailed from the medical care provider's place of business after the saliva sample, menstrual blood sample or menstrual effluent sample is collected from the patient during an appointment (430). The code uniquely identifying the saliva sampling kit (or menstrual effluent sampling kit or menstrual blood sampling kit) may be provided by either the medical care provider or patient via a web portal (see e.g. 510 and 615). In some embodiments, the medical care provider's unique pre-assigned code is provided by the patient alongside the code uniquely identifying the saliva sampling kit or menstrual effluent (e.g. blood) sampling kit (see e.g. 510, 515, 520). In some embodiments, the patient's unique identifier code is provided by the medical care provider alongside the code uniquely identifying the saliva sampling kit or menstrual effluent (e.g., menstrual blood) sampling kit (see e.g. 615). Sample, patient, and medical care provider information then may be associated. In some embodiments, this involves associating, in a second database, the code which uniquely identifies the subject with the code which uniquely identifies the saliva sampling kit (or menstrual effluent sampling kit or menstrual blood sampling kit) and the code which uniquely identifies the subject's medical care provider. Such association may be via any suitable method. In some embodiments, the submission of the medical care provider's identification code and the saliva sample identification code (or menstrual effluent sample identification code or menstrual blood sample identification code) by the subject via a web interface specific to the subject provides the information to associate the medical care provider's, subject, and saliva/menstrual effluent sample kit (or menstrual blood sample kit) identification codes (see e.g. 616 and 621 for the provider side and 525 and 530 for the patient's side). In other embodiments, the submission of the subject's identification code and the saliva sample (or menstrual effluent or blood) identification code by the medical care provider via a web interface specific to the medical care provider (e.g. physician) provides the information to associate the medical care provider's, subject, and saliva sample kit (or menstrual effluent or blood sample kit) identification codes (see e.g. 620). In yet other embodiments, such information is entered directly into a database. In some embodiments, any of the materials that are received by a web interface are provided via fax.

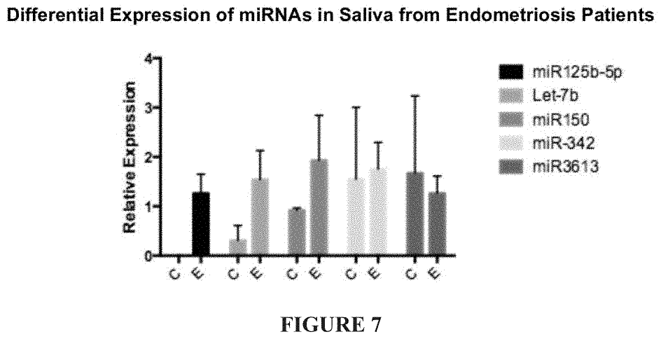

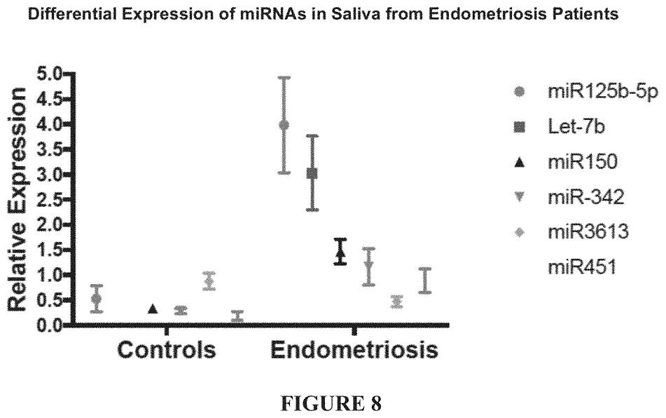

In some cases, the saliva sample, menstrual blood sample, or menstrual effluent sample in the received saliva sampling kit or menstrual effluent sampling kit is then processed to determine an expression level of at least one miRNA, at least one ncRNA, or at least one miRNA in combination with at least one ncRNA that is not an miRNA. An exemplary protocol for processing of the saliva, menstrual blood or menstrual effluent sample is provided in Example 3. In some embodiments, this processing is done directly by applicant in a CLIA-certified lab. In other embodiments, this testing is done indirectly by applicant by submitting the sample to a third-party CLIA-certified lab, and the miRNA, ncRNA, and/or ncRNA that is not an miRNA expression results are received by applicant from the third-party CLIA-certified lab. miRNAs and ncRNAs for which the expression levels are measured may be any of the miRNAs and ncRNAs identified in this disclosure (e.g. those associated with endometriosis). Exemplary biomarkers showing differential regulation in endometriosis are shown in FIGS. 7 and 8. In some embodiments, the one or more miRNAs is selected from the group consisting of miR-125, miR-150, miR-342, miR-145, miR-143, miR-500, miR-451, miR-18, miR-214, miR-126, miR-6755, miR-3613, miR-553, and miR-4668, and any combination thereof. An expression level of at least one miRNA or ncRNA associated with other diseases/disorders may also be ascertained. In some embodiments, the one or more miRNA is a cell-free miRNA. In some embodiments, the one or more ncRNA (e.g., ncRNA that is not an miRNA) is a cell-free ncRNA. In some embodiments, the one or more miRNA is a cell-associated miRNA. In some embodiments, the one or more ncRNA (e.g., ncRNA that is not an miRNA) is a cell-associated ncRNA.

Whether the processing of the saliva sample is performed by applicant or a third-party, the expression level of the at least one miRNA, at least one ncRNA, or at least one miRNA/ncRNA that is not an miRNA from the processed saliva, menstrual blood or menstrual effluent sample is entered into a third database and the saliva sampling kit identification code is used to associate the expression level results with the subject's unique identifier code and the subject's medical care provider's unique identifier code via the association created previously. Additional processing of the expression level information may be performed, such as use of a classification algorithm to assign a diagnostic indication to the expression level results; such additional processing is likewise associated with the expression level data and the physician's and patient's identifying codes. In some embodiments, the clinical indication assigned is endometriosis.

By virtue of the association of the expression level results with both the medical care provider's and subject's unique identifier codes, the expression level results (and any additional processing, such as the assignation of a clinical indication) are accessible by both the medical care provider (e.g. physician) and the subject (e.g. via their respective specific web portals).

Identification and Treatment of Endometriosis with GnRH Antagonists or Agonists

In some cases, the present disclosure provides methods of conducting a diagnostic test for endometriosis (e.g., refractory endometriosis) and managing treatment thereof via the diagnostic results. Given the limitations of existing treatment regimens for endometriosis, there is a need for better modes of personalized management of treatment regimens. First-line treatments for endometriosis either manage pain without affecting the disease process per se (e.g. NSAIDS), or ultimately prove ineffective in certain patients (e.g., progestins, which are ineffective in suppressing endometriosis in a subgroup of women whose endometrial tissue does not respond normally to progesterone). Second line therapies, such as GnRH agonists or antagonists, are associated with varying degrees of unpleasant side effects. Accordingly, there is a need for improved monitoring of endometriosis for identification of appropriate treatment regiments or management of dosing for existing treatment regimens.

In some cases, this disclosure includes methods of identifying, detecting, and/or treating endometriosis (e.g., refractory endometriosis) in a subject (e.g., a subject receiving progestin therapy). In some embodiments, this method comprises first (a) obtaining a fluid sample from the subject, wherein the fluid sample comprises ribonucleic acids and the subject is receiving progestin therapy for endometriosis. The fluid sample may be any body fluid, but preferred sweat, saliva, tears, urine, blood, plasma, serum, vaginal fluid, cervico-vaginal fluid, whole blood, blood serum, blood plasma, menstrual effluent, menstrual blood, spinal fluid, or pulmonary fluid. In some embodiments, the fluid sample is saliva. In other embodiments the fluid sample is menstrual effluent or menstrual blood. In some embodiments, the subject is receiving a progestin-treatment regimen, such as dydrogesterone, medroxyprogesterone acetate, depot medroxyprogesterone acetate, norethisterone, or an oral contraceptive pill. In other embodiments, the subject is not receiving a progestin treatment therapy. In some embodiments, the subject is experiencing symptoms associated with endometriosis (e.g. dysmennorhea, pain with bowel movements or urination, or excessive bleeding) prior to obtaining the fluid sample. In other embodiments, the subject is not experiencing symptoms associated with endometriosis.

In some embodiments, the method may further comprise (b) determining an expression level of at least one miRNA, at least one ncRNA, or at least one miRNA in combination with at least one ncRNA that is not an miRNA corresponding to the ribonucleic acids from the saliva sample (or menstrual effluent or menstrual blood) from the subject, wherein the at least one miRNA or ncRNA, or combination thereof, is associated with endometriosis. In some embodiments, the at least one miRNA or ncRNA is associated with endometriosis. In other embodiments, the at least one miRNA or ncRNA is associated with hormone-refractory endometriosis. In some embodiments, the at least one miRNA or ncRNA is selected from the group consisting of let-7a, let-7b, let-7c, let-7d, let-7e, let-7f, miR-135a, miR-135b, miR-18a, miR-125b, miR-143, miR-145, miR-150, miR-342, miR-451a, miR-500a, miR-3613, and miR-6755, or any combination thereof. In some embodiments, the at least one ncRNA is also associated with endometriosis.

The method may next comprise (c) diagnosing endometriosis in the subject based on the expression level of the at least one miRNA or at least one ncRNA determined from the fluid sample. In some embodiments, the presence of a single miRNA or ncRNA denotes endometriosis (e.g., when only a single miRNA associated with endometriosis is measured). In other embodiments, diagnosis of endometriosis is performed based on the expression levels of multiple miRNAs or ncRNAs. Such a diagnosis can involve the use of a computer implemented classification algorithm to assign a likelihood of endometriosis based on the expression levels of multiple miRNAs or ncRNAs.

Finally, (d) administration of an initial dose regimen of a GnRH antagonist or agonist (e.g., Elagolix) to the subject may be performed in order to treat the endometriosis diagnosed herein based on the expression level of the one or more miRNA or ncRNA. A variety of antagonists of GnRH suitable for clinical administration, both peptide (goserelin acetate, buserelin, histrelin, deslorelin, nafarelin, and triptorelin, leuproreolin) and non-peptide (Elagolix/ABT-620, NBI-56418, see for e.g. Taylor et al. N Engl J Med. 2017 Jul. 6; 377(1):28-40), are available for second-line treatment of endometriosis in individuals with refractory endometriosis. In some embodiments, the fluid sample collection and diagnosis described above are performed after a defined period of time (e.g. 1 month, 6 month, or 1 year) and the initial dose of the GnRH antagonist or agonist is adjusted downward when endometriosis is not detected. In some embodiments, the fluid sample collection and diagnosis described above are performed after a defined period of time (e.g. 1 month, 6 month, or 1 year) and the initial dose of the GnRH antagonist or agonist is adjusted upward when endometriosis is detected. In some embodiments, the fluid sample collection and diagnosis described above are performed after a defined period of time (e.g. 1 month, 6 month, or 1 year) and administration of the GnRH antagonist or agonist is terminated when endometriosis is not detected.

In some embodiments, an endometriosis treatment (e.g., GnRH antagonist, Elagolix) is administered at a particular dose in order to treat, prevent or reduce symptoms of endometriosis. In some cases, the dose of the endometriosis treatment (e.g., GnRH antagonist, Elagolix) is at least 10 mg, at least 15 mg, at least 20 mg, at least 25 mg, at least 50 mg, at least 75 mg, at least 100 mg, at least 125 mg, at least 150 mg, at least 175 mg, at least 200 mg, at least 225 mg, at least 250 mg, at least 275 mg, at least 300 mg, at least 325 mg, at least 350 mg, at least 375 mg, at least 400 mg, or more. In some embodiments, the dose of the endometriosis treatment (e.g., GnRH antagonist, Elagolix) is less than 10 mg, less than 15 mg, less than 20 mg, less than 25 mg, less than 50 mg, less than 75 mg, less than 100 mg, less than 125 mg, less than 150 mg, less than 175 mg, less than 200 mg, less than 225 mg, less than 250 mg, less than 275 mg, less than 300 mg, less than 325 mg, less than 350 mg, less than 375 mg, or less than 400 mg.

The endometriosis treatment (e.g., GnRH antagonist, Elagolix) may be administered at any number of frequencies including once per day, once every other day, twice per day, etc. In some cases, the endometriosis treatment is administered once a day at a dose of between 150 mg −200 mg g (e.g., 150 mg, 175 mg, 200 mg) or 150-300 mg (e.g., 150 mg, 175 mg, 200 mg, 250 mg, 275 mg, 300 mg). In some cases, the endometriosis treatment is administered twice a day at a dose of between 150 mg −200 mg g (e.g., 150 mg, 175 mg, 200 mg) or 150-300 mg (e.g., 150 mg, 175 mg, 200 mg, 250 mg, 275 mg, 300 mg). In some particular examples, the subject is administered a dose in a range of 150 mg once daily to 200 mg twice daily.

In some cases, the detection of endometriosis (e.g., through identifying ncRNA or miRNA profiles) or detection of a reduction in endometriosis may lead to, or provide motivation for increasing the dose of endometriosis treatment (e.g., GnRH antagonist, Elagolix) administered to the subject. For example, if a subject is administered 100 mg once daily (or 150 mg once daily), the dose may be increased, in some cases to 175 mg twice daily, 125 mg twice daily, 100 mg twice daily, 75 mg twice daily, 50 mg twice daily, 25 mg twice daily, 200 mg once daily, 175 mg once daily, 150 mg once daily, 125 mg once daily, or any combination thereof. In some cases, the dose is progressively increased over time. In some cases, the dose is increased by 5%, 10%, 15%, 25%, 50%, 75%, 100%. In some cases, the subject may then be monitored again for ncRNA or miRNA levels associated with endometriosis, and the dose adjusted again if necessary.

In some cases, the detection of endometriosis (e.g., through identifying ncRNA or miRNA profiles) or detection of a reduction in endometriosis may lead to, or provide motivation for lowering the dose of endometriosis treatment (e.g., GnRH antagonist, Elagolix) administered to the subject. For example, if a subject is taking the treatment 200 mg, twice daily, the dose may be lowered, in some cases to 175 mg twice daily, 125 mg twice daily, 100 mg twice daily, 75 mg twice daily, 50 mg twice daily, 25 mg twice daily, 200 mg once daily, 175 mg once daily, 150 mg once daily, 125 mg once daily, 100 mg once daily, 75 mg once daily, or 50 mg once daily, or any combination thereof. In some cases, the dose is progressively reduced or lowered over time. In some cases, the dose is reduced by 5%, 10%, 15%, 25%, 50%, 75%, 100%. In some cases, the subject may then be monitored again for ncRNA or miRNA levels associated with endometriosis, and the dose adjusted again if necessary.

The endometriosis treatment may be administered by a number of routes, e.g., orally, intravenously. In particular, the treatment is administered orally. For example, the treatment may be administered as a pill, tablet, gel tablet, caplet, liquid, or any other known mode of administration for the treatment.

In some cases, the administration of the endometriosis treatment (e.g., GnRH antagonist, Elagolix) may lead to partial estrogen suppression in the subject (e.g., a dose of 150 mg once a day may have such effect). In some cases, the administration of the of endometriosis treatment (e.g., GnRH antagonist, Elagolix) may lead to complete or near complete estrogen suppression in the subject (e.g, a dose of 200 mg twice daily may have such effect).

In some methods, expression levels are determined at intervals in a particular patient (e.g., by monitoring). Preferably, the monitoring is conducted by serial minimally-invasive or non-invasive tests such as blood draws, saliva collection, menstrual blood, or menstrual effluent collection. The monitoring may occur at different intervals, for example the monitoring may be hourly, daily, weekly, monthly, yearly, or some other time period, such as twice a month, three times a month, every two months, every three months, every six months, every nine months, every other year, etc.

Such methods can provide a series of values changing over time indicating whether the aggregate miRNA or ncRNA levels in a particular patient are more like the expression levels in patients having endometriosis (the endometriosis “signature”). Movement in value toward or away from the endometriosis signature can provide an indication whether an existing progestin or GnRH therapy is working, whether the progestin or GnRH therapy should be changed or whether a laparoscopy or ultrasound test should be performed.

### EXAMPLES

### Example 1: Saliva microRNAs as Diagnostic Markers for Endometriosis

Step 1: RNA Extraction from Saliva

Saliva samples (200 μL) were collected from both a female control group and a female group with clinically-verified endometriosis and transferred to 1.5 mL tubes. RNase free water was added to samples with volumes less than 200 μL in order to bring the total sample volume to 200 μL. 1 mL of QIAzol Lysis Reagent (Qiagen) was added to the sample. The tube was vortexed briefly, and the sample was allowed to incubate at room temperature for five minutes. Then, 200 μL of chloroform was added to the lysate and vortexed for approximately 15 seconds. The sample mixture was then incubated for two minutes at room temperature and centrifuged at 12,000×g for fifteen minutes in a cold room (approximately 4° C.). Approximately 560 μL of the aqueous phase was transferred to a new 1.5 mL tube. 840 μL of 100% ethanol was added to the 560 μL of aqueous phase to obtain a total volume of 1400 μL. 700 μL of the mixture was then transferred into a RNeasy MinElute spin column with 2 mL collection tube. The spin column with collection tube was centrifuged at 9,000×g for 15 seconds. The flow-through was discarded and the remaining 700 μL of mixture was transferred to the spin column with collection tube and again centrifuged at 9,000×g for 15 seconds. The flow-through was discarded and 700 μL of buffer RWT was added to each spin column and then centrifuged at 9,000×g for 15 seconds. The flow-through was discarded and 500 μL of buffer RPE was added to each spin column and then centrifuged at 9,000×g for 15 seconds. The flow-through was discarded and another 500 μL of buffer RPE was added to the spin column and again centrifuged at 9,000×g for 15 seconds. Then, 500 μl of 80% ethanol was added to the spin column and centrifuged at 9,000×g for 2 minutes. The flow-through and collection tube were subsequently discarded and the spin column was transferred to a new 2 mL collection tube. The lid of the spin column was left open and then centrifuged at 12,000×g for 5 minutes to dry the membrane. The spin column was then placed in a 1.5 mL tube. 14 μL of RNase-free water was added to the spin column and it was centrifuged at 12,000×g for 1 minute to elute total RNA. The spin column was discarded and the RNA was stored at −80° C.

Step 2: Preparation of cDNA

The TaqMan Advanced miRNA cDNA Synthesis Kit was used to prepare cDNA (ThermoFisher, catalog number: A28007) in four sequential steps: A, B, C, and D.

Step A was performed with the following contents in each reaction: 0.5 μL 10× Poly A buffer; 0.5 μL 10 mM ATP; 0.3 μL Poly A enzyme, 5 U/μL; 1.7 μL RNase-free water; 2.0 μL sample. The plate or tube was sealed and vortexed briefly. The plate or tube was centrifuged to spin down the contents and eliminate any air bubbles. The plate or tube was placed into a thermal cycler and incubated with the following settings:

### 1. Polyadenylation at 37° C. for 45 Minutes.

2. Stop reaction at 65° C. for 10 minutes.

### 3. Hold at 4° C.

Step B was performed with the following contents in each reaction: 3.0 μL 5×DNA Ligase Buffer; 4.5 μL 50% PEG 8000; 0.6 μL 25× Ligation Adaptor; 1.5 μL RNA Ligase; 0.4 μLRNase-free water. The Ligation Reaction Mix was vortexed to thoroughly mix the contents and then centrifuged briefly to spin down the contents and eliminate air bubbles. 10 μL of the Ligation Reaction Mix was transferred to each well of the reaction plate or each reaction tube containing the poly(A) tailing reaction product. The reaction plate or tubes were sealed, then vortexed briefly or shaken (1,900 RPM for 1 minute with an Eppendorf™ MixMate™) to thoroughly mix the contents. The reaction plate or tubes were centrifuged briefly to spin down the contents. The plate or tube was placed into the thermal cycler.

Step C was performed with the following contents in each reaction: 6 μL 5×RT Buffer; 1.2 μL dNTP Mix (25 mM each); 1.5 μL 20× Universal RT Primer; 3 μL 10× RT Enzyme Mix; 3.3 μL RNase-free water. The RT Reaction Mix was vortexed to thoroughly mix the contents, and then centrifuged briefly to spin down the contents and eliminate air bubbles. 15 μl of the RT Reaction Mix was transferred to each well of the reaction plate or each reaction tube containing the adaptor ligation reaction product. The total volume was 30 μl per well or tube. The reaction plate or tubes were then sealed and vortexed briefly to thoroughly mix the contents. The reaction plate or tubes were then centrifuged briefly to spin down the contents. The plate or tube was placed in the thermal cycler and incubated with the following settings:

1. Reverse transcription at 42° C. for 15 minutes
2. Stop reaction at 85° C. for 5 minutes

### 3. Hold at 4° C.

Step D was performed with the following contents in each reaction: 25 μL 2× miR-Amp Master Mix; 2.5 μL 20× miR-Amp Primer Mix; 17.5 μL RNase-free water. The miR-Amp Reaction Mix was vortexed to thoroughly mix the contents, then centrifuged briefly to spin down the contents and eliminate air bubbles. 45 μL of the miR-Amp Reaction Mix was transferred to each well of a new reaction plate or reaction tube. 5 μL of the RT reaction product was added to each reaction well or reaction tube. The total volume in each well or tube was 50 μL. The reaction plate or tubes were sealed and then vortexed briefly to thoroughly mix the contents. The reaction plate or tubes were then briefly centrifuged to spin down to mix the contents. The reaction plate or tubes were placed into a thermal cycler and then incubated using the following settings, MAX ramp speed, and standard cycling:

1. Enzyme activation at 95° C. for 5 minutes, 1 cycle
2. Denature at 95° C. for 3 seconds, 14 cycles
3. Anneal/Extend at 60° C. for 30 seconds, 14 cycles
4. Stop reaction at 99° C. for 10 minutes, 1 cycle.

### 5. Hold at 4° C.

Step 3: Amplification of microRNAs

RT-PCR Protocol:

### 1. 95° C. for 3 min

### 2. 95° C. for 15 s

### 3. 59° C. for 5 s

### 4. 72° C. for 55 s

5. Repeat 2-4 steps for 39 cycles

### 6. Melting Curve at 55° C. for 10 s

### 7. 95° C. for 5 s

### 8. Hold at 4° C.

The relative expression of saliva miRNAs that were differentially expressed between the control group and the endometriosis group are shown in FIG. 7 and FIG. 8, in which FIG. 7 depicts results from an initial study and FIG. 8 depicts a repeat study with higher N. The data in FIG. 7 represents fifteen samples in each group, while the data in FIG. 8 represents an updated experiment with data from 80 samples split 50:50 between endometriosis and control groups. Levels of miR125b-5p, Let-7b, and miR-150 all display upregulation in the saliva samples of the endometriosis group (E) relative to the control group (C), with miR125b-5p showing the highest fold/confidence upregulation. Levels of miR-342 and miR-451 additionally shows upregulation in (E) vs (C). In contrast, levels of miR-3613 show downregulation in (E) vs (C) (see e.g. FIG. 8).

### Example 2: Detection, Diagnosis and Treatment of Endometriosis (Prophetic Example)

A blood, blood plasma, blood serum, menstrual blood, menstrual effluent, urine, or saliva sample is taken from a female patient with symptoms of endometriosis. The quantity of a signature of microRNA associated with endometriosis (for example, let-7a, let-7b, let-7c, let-7d, let-7e, let-7f, miR-135a, miR-135b, miR-18a, miR-125b, miR-143, miR-145, miR-150, miR-342, miR-451a, miR-500a, miR-3613, or miR-6755, or any combination thereof) is then determined in the sample, and the patient is diagnosed with endometriosis if the signature is within a certain window indicating the presence of endometriosis. The patient is treated with a therapeutically effective dose of a GnRH antagonist or agonist therapy (e.g., Elagolix). The compound causes a reduction in the symptoms of endometriosis. After one month of treatment, six months of treatment, and one year of treatment, the patient is assessed for levels of a microRNA signature associated with endometriosis. If the microRNA signature associated with endometriosis indicates the presence of endometriosis, the dose of the GnRH agonist or antagonist therapy (e.g., Elagolix) is adjusted upward, and the treatment/testing process is repeated until biomarkers indicate the absence of endometriosis.

### Example 3: Detection, Diagnosis and Treatment of Treatment-Resistant Endometriosis (Prophetic Example)

A blood, blood plasma, blood serum, menstrual blood, menstrual effluent, urine, or saliva sample is taken from a female patient with previously diagnosed endometriosis who is currently receiving a progestin-based therapy (e.g. dydrogesterone, medroxyprogesterone acetate, depot medroxyprogesterone acetate, norethisterone, or an oral contraceptive pill), but does not appear to be improving. In some cases, the patient may have refractory endometriosis. The quantity of a microRNA signature associated with endometriosis (for example, let-7a, let-7b, let-7c, let-7d, let-7e, let-7f, miR-135a, miR-135b, miR-18a, miR-125b, miR-143, miR-145, miR-150, miR-342, miR-451a, miR-500a, miR-3613, or miR-6755, or any combination thereof) is then determined in the sample and compared to a reference value associated with remission of endometriosis. This provides guidance for administration of a GnRH antagonist (goserelin acetate, buserelin, histrelin, deslorelin, nafarelin, and triptorelin, leuproreolin, or Elagolix) or a GnRH agonist. The initial dose of the GnRH antagonist or agonist can be adjusted downward based on future negative microRNA testing, or upward if future testing indicates the initial dose is not sufficient for suppression of endometriosis. Collation of microRNA biomarker levels over time provides ongoing evidence of the effectiveness of the GnRH antagonist or agonist.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of identifying and treating endometriosis in a female subject, comprising:
(a) obtaining a fluid sample from the female subject, wherein the fluid sample comprises ribonucleic acids (RNA);
(b) determining an expression level of at least one miRNA or at least one non-coding RNA (ncRNA) from the fluid sample from the subject, wherein the at least one miRNA or the at least one ncRNA is associated with endometriosis;
(c) diagnosing endometriosis in the subject based on the expression level of the at least one miRNA or the at least one ncRNA; and
(d) administering an initial dose regimen of a Gonadotropin-releasing hormone (GnRH) antagonist to the subject in order to treat the endometriosis diagnosed in the subject in (c).

2. The method of claim 1, wherein the fluid sample comprises at least one miRNA.

3. The method of claim 1, wherein the fluid sample is blood, saliva, menstrual blood, or menstrual effluent.

4. The method of claim 1, wherein the female subject is receiving treatment for endometriosis, and the endometriosis diagnosed and treated is refractory endometriosis.

5. The method of claim 4, wherein the treatment is progestin therapy.

6. The method of claim 5, wherein the progestin therapy is dydrogesterone, medroxyprogesterone acetate, depot medroxyprogesterone acetate, norethisterone, or an oral contraceptive pill.

7. The method of claim 1, wherein the subject is experiencing symptoms associated with endometriosis.

8. The method of claim 7, wherein the subject is experiencing one or more of dysmennorhea, pain with bowel movements or urination, or excessive bleeding.

9. The method of claim 1, wherein the subject is not experiencing symptoms associated with endometriosis.

10. The method of claim 1, wherein the at least one miRNA is selected from the group consisting of let-7a, let-7b, let-7c, let-7d, let-7e, let-7f, miR-135a, miR-135b, miR-18a, miR-125b, miR-143, miR-145, miR-150, miR-342, miR-451a, miR-500a, miR-3613, and miR-6755, or any combination thereof.

11. The method of claim 1, wherein the at least one miRNA is selected from the group consisting of let-7c, let-7d, let-7f, miR-18a, miR-125b, miR-143, miR-150, miR-342, miR-451a, miR-500a, miR-3613, and miR-6755, or any combination thereof.

12. The method of claim 1, wherein the at least one miRNA is selected from the group consisting of let-7a, let-7b, let-7c, let-7d, let-7e, let-7f, mir135a, and mir135b, or any combination thereof.

13. The method of claim 1, wherein the at least one miRNA is selected from the group consisting of miR-125b, miR-150, miR-342, miR-451a, miR-3613, and let-7b, or any combination thereof.

14. The method of claim 1, wherein the at least one miRNA is selected from the group consisting of miR-150, 451a, and 3613, or any combination thereof.

15. The method of claim 1, further comprising repeating (a)-(c) and adjusting the initial dose regimen of the GnRH antagonist when endometriosis is not detected.

16. The method of claim 1, further comprising repeating (a)-(c) and adjusting the initial dose regimen of the GnRH antagonist when endometriosis is detected.

17. The method of claim 1, further comprising repeating (a)-(c) and terminating administration of the GnRH antagonist when endometriosis is not detected.

18. The method of claim 1 comprising repeating (a)-(c) every 1 month, 6 months, or 1 year.

19. The method of claim 1, wherein the GnRH antagonist is goserelin acetate, buserelin, histrelin, deslorelin, nafarelin, and triptorelin, leuproreolin, or Elagolix.

20. The method of claim 3, wherein the sample is menstrual blood or menstrual effluent and the menstrual blood or menstrual effluent is collected by the subject using a menstrual cup.

21. The method of claim 3, wherein the sample is saliva and the saliva was collected by the subject using a home saliva sampling kit.

22. A method of identifying and treating endometriosis in a female subject, comprising;
(a) receiving information characterizing an expression level of at least one miRNA or non-coding RNA (ncRNA) from a fluid sample from the female subject;
(b) diagnosing endometriosis in the subject based on the expression level of the at least one miRNA or the at least one ncRNA from the fluid sample from the female subject; and
(c) administering an initial dose regimen of a Gonadotropin-releasing hormone (GnRH) antagonist to the female subject in order to treat the endometriosis diagnosed in the female subject in (b).

23. The method of claim 22, wherein the at least one miRNA is selected from the group consisting of let-7a, let-7b, let-7c, let-7d, let-7e, let-7f, miR-135a, miR-135b, miR-18a, miR-125b, miR-143, miR-145, miR-150, miR-342, miR-451a, miR-500a, miR-3613, and miR-6755, or any combination thereof.

24. The method of claim 22, further comprising repeating (a)-(b) and adjusting the initial dose regimen of the GnRH antagonist when endometriosis is not detected.

25. The method of claim 22, further comprising repeating (a)-(b) and adjusting the initial dose regimen of the GnRH antagonist when endometriosis is detected.

26. The method of claim 22, further comprising repeating (a)-(b) and terminating administration of the GnRH antagonist when endometriosis is not detected.

27. The method of claim 22 comprising repeating (a)-(b) every 1 month, 6 months, or 1 year.

28. The method of claim 22, wherein the GnRH antagonist is goserelin acetate, buserelin, histrelin, deslorelin, nafarelin, triptorelin, leuproreolin, or Elagolix.

29. The method of claim 22, wherein the fluid sample is blood, plasma, serum, saliva, menstrual blood, or menstrual effluent.

30. A method of treating endometriosis in a female subject, comprising administering to the female subject an initial dose regimen of a Gonadotropin-releasing hormone (GnRH) antagonist, wherein a fluid sample from the female subject has a level of at least one miRNA or at least one ncRNA associated with endometriosis.

31. The method of claim 30, wherein the fluid sample is blood, plasma, serum, saliva, menstrual blood or menstrual effluent.

32. The method of claim 30, wherein the at least one miRNA is selected from the group consisting of let-7a, let-7b, let-7c, let-7d, let-7e, let-7f, miR-135a, miR-135b, miR-18a, miR-125b, miR-143, miR-145, miR-150, miR-342, miR-451a, miR-500a, miR-3613, and miR-6755, or any combination thereof.

33. The method of claim 30, wherein the GnRH antagonist is goserelin acetate, buserelin, histrelin, deslorelin, nafarelin, and triptorelin, leuproreolin, or Elagolix.

34. The method of claim 30, wherein the endometriosis is refractory endometriosis.

35. The method of claim 30, wherein the female subject is receiving a progestin therapy and the endometriosis is refractory endometriosis.

36. A method of treating endometriosis in a subject in need thereof comprising:
administering to the subject in need thereof an initial dose of a Gonadotropin-releasing hormone (GnRH)-antagonist;
monitoring the level of at least one miRNA or at least one non-coding RNA (ncRNA) associated with endometriosis in the subject in need thereof over time; and
adjusting the initial dose of said GnRH-antagonist when the level of the at least one miRNA or the at least one ncRNA associated with endometriosis increases or decreases over time.

37. The method of claim 36, wherein the GnRH antagonist is goserelin acetate, buserelin, histrelin, deslorelin, nafarelin, and triptorelin, leuproreolin, or Elagolix.

38. The method of claim 36, wherein the at least one miRNA is selected from the group consisting of let-7a, let-7b, let-7c, let-7d, let-7e, let-7f, miR-135a, miR-135b, miR-18a, miR-125b, miR-143, miR-145, miR-150, miR-342, miR-451a, miR-500a, miR-3613, and miR-6755, or any combination thereof.

39. The method of claim 36, wherein monitoring comprises:
(a) determining an expression level of at least one miRNA or the at least one ncRNA from a fluid sample from the subject; or
(b) receiving information characterizing the expression level of at least one miRNA or the at least one ncRNA from a fluid sample from the female subject.

40. The method of claim 38, comprising adjusting the initial dose of GnRH antagonist when the levels of at least one of miR-3613 or let-7b decrease over time.

41. The method of claim 38, comprising adjusting the initial dose of GnRH when the levels of at least one of miR-125b, miR-150, miR-342, or miR-451a increase over time.

42. The method of claim 36, wherein the time over which the level of the at least one miRNA or the at least one ncRNA increases or decreases is 1 month, 6 months, or 1 year.

43. A method of detecting miRNA or non-coding RNA (ncRNA) in a female subject suspected of having endometriosis, comprising detecting at least one miRNA or at least one ncRNA from a fluid sample from the female subject suspected of having endometriosis, wherein the fluid sample comprises menstrual effluent or menstrual blood.

44. The method of claim 43, further comprising administering an initial dose regimen of a treatment for endometriosis to the female subject suspected of having endometriosis.

45. The method of claim 44, wherein the treatment for endometriosis comprises a GnRH antagonist.

46. The method of claim 43, wherein the at least one miRNA is selected from the group consisting of let-7a, let-7b, let-7c, let-7d, let-7e, let-7f, miR-135a, miR-135b, miR-18a, miR-125b, miR-143, miR-145, miR-150, miR-342, miR-451a, miR-500a, miR-3613, and miR-6755, or any combination thereof.

47. The method of claim 43 comprising repeating the detecting of the at least one miRNA or the at least one ncRNA every 1 month, 6 months, or 1 year.

48. The method of claim 43, further comprising diagnosing endometriosis in the subject suspected of having endometriosis based on an expression level of the at least one miRNA or the at least one ncRNA from the fluid sample from the female subject and administering to the female subject a treatment for endometriosis.

49. A method of treating endometriosis in a female subject, comprising administering to the female subject an initial dose regimen of an endometriosis treatment when a menstrual blood or menstrual effluent sample from the female subject has a level of at least one miRNA or at least one ncRNA associated with endometriosis.

50. The method of claim 49, wherein the at least one miRNA is selected from the group consisting of let-7a, let-7b, let-7c, let-7d, let-7e, let-7f, miR-135a, miR-135b, miR-18a, miR-125b, miR-143, miR-145, miR-150, miR-342, miR-451a, miR-500a, miR-3613, and miR-6755, or any combination thereof.

51. The method of claim 49, wherein the endometriosis is refractory endometriosis.

52. The method of claim 49, wherein the female subject is receiving a progestin therapy and the endometriosis is refractory endometriosis.

53. A method of conducting a diagnostic test on a subject and providing results to a medical care provider of the subject, comprising:
(a) providing to the subject a saliva, menstrual blood, or menstrual effluent sampling kit, wherein the saliva, menstrual blood, or menstrual effluent sampling kit comprises: (i) a saliva, menstrual blood, or menstrual effluent recovery and collection device; and (ii) a code which uniquely identifies the saliva, menstrual blood, or menstrual effluent recovery and collection device;
(b) assigning, in a first database, a code which uniquely identifies the subject;
(c) receiving separately from the subject (i) a saliva, menstrual blood, or menstrual effluent sample in the saliva, menstrual blood, or menstrual effluent; (ii) the code which uniquely identifies the saliva, menstrual blood, or menstrual effluent sampling kit; and (iii) a pre-assigned code which uniquely identifies the medical care provider of the subject;
(d) associating, in a second database, the code which uniquely identifies the subject with the code which uniquely identifies the saliva, menstrual blood, or menstrual effluent and the pre-assigned code which uniquely identifies the medical care provider of the subject;
(e) processing the saliva sample in the saliva, menstrual blood, or menstrual effluent to determine an expression level of at least one miRNA or at least one ncRNA; and
(f) entering the expression level of at least one miRNA or the at least one ncRNA from the processed saliva, menstrual blood, or menstrual effluent sample into a third database and associating the expression level of at least one miRNA with the subject and the medical care provider of the subject via the association created in (d), wherein the expression level of at least one miRNA or of the at least one ncRNA in the database is accessible via a web-based interface by the subject and the medical care provider of the subject.

54. The method of claim 53, wherein the first, second, and third databases are a single database.

55. The method of claim 53, wherein the first, second, and third databases are separate databases.

56. The method of claim 53, wherein (a) comprises mailing the saliva sampling kit to the subject at a home address or preferred mailing address of the subject.

57. The method of claim 53, wherein (a) comprises mailing the saliva, menstrual blood, or menstrual effluent sampling kit to the medical care provider of the subject.

58. The method of claim 53, wherein (a) comprises billing the saliva, menstrual blood, or menstrual effluent testing kit to a credit card of the subject.

59. The method of claim 53, wherein the code that uniquely identifies the saliva, menstrual blood, or menstrual effluent sampling kit is provided by the medical care provider of the subject via a web interface.

60. The method of claim 53, wherein the code that uniquely identifies the saliva, menstrual blood, or menstrual effluent sampling kit is provided by the subject via a web interface.

61. The method of claim 53, wherein receiving the saliva, menstrual blood, or menstrual effluent sampling kit from the subject in (c) comprises receiving the saliva, menstrual blood, or menstrual effluent sampling kit via mail from the subject's home address or preferred mailing address.

62. The method of claim 53, wherein receiving the saliva sample in the saliva, menstrual blood, or menstrual effluent sampling kit from the subject in (c) comprises receiving the saliva, menstrual blood, or menstrual effluent sampling kit via mail from a workplace address of the medical care provider of the subject.

63. The method of claim 53, further comprising providing a clinical indication based on the expression level of the at least one miRNA or the at least one ncRNA, wherein the clinical indication is also accessible via the web-based interface by the subject and the medical care provider of the subject.

64. The method of claim 53, wherein the clinical indication is endometriosis.

65. The method of claim 53, wherein the processing the saliva, menstrual blood, or menstrual effluent sample in the saliva, menstrual blood, or menstrual effluent sampling kit to determine an expression level of the at least one miRNA or the at least one ncRNA in (e) comprises sending the saliva sampling kit to a third party diagnostic laboratory to determine an expression level of the at least one miRNA or the at least one ncRNA.

66. A method of conducting a diagnostic test for endometriosis on a subject and providing results to the subject and the medical care provider of the subject, comprising:
(a) assigning, in a first database, a code which uniquely identifies the subject;
(b) receiving from the subject (i) a stabilized fluid sample; (ii) the code which uniquely identifies the stabilized fluid sample; and (iii) a pre-assigned code which uniquely identifies the medical care provider of the subject;
(c) associating, in a second database, the code which uniquely identifies the subject with the code which uniquely identifies the fluid sample and the pre-assigned code which uniquely identifies the medical care provider of the subject;
(d) processing the fluid sample to determine an expression level of at least one miRNA or at least one non-coding RNA (ncRNA); and
(e) entering the expression level of the at least one miRNA or the at least one ncRNA from the processed fluid sample into a third database and associating the expression level of at least one miRNA or the at least one ncRNA with the subject and the medical care provider of the subject via the association created in (d), wherein the expression level of at least one miRNA or the at least one ncRNA in the database is accessible via a web-based interface by the subject and the medical care provider of the subject.

67. The method of claim 66, wherein the fluid sample is a saliva, menstrual blood, or menstrual effluent sample.

68. The method of claim 67, wherein the fluid sample is a menstrual blood or menstrual effluent sample.

69. The method of claim 68, wherein the menstrual blood or menstrual effluent sample is stabilized by spotting and drying on paper.

70. The method of claim 68, wherein the menstrual blood or menstrual effluent sample is stabilized by addition of RNase inhibitor.
